SEQUENCE LISTING (16 sequences)

SEQ ID NO: 1 - protein, 4 aa, Arabidopsis thaliana
WRKY

SEQ ID NO: 2 - DNA, 7 bp, Arabidopsis thaliana
  misc_feature 7: n = C or T
tttgacn

SEQ ID NO: 3 - DNA, 1101 bp, Arabidopsis thaliana
atgagaccgattcaatcgcctccaggagtttccgttccggtgaaaagccgtccccgtcgccgtcctgatcttaccttaccgcttcctcaacgcgatgtttctctcgctgtacctcttcctctcccacctacttccggtggttccggtggctctagtggatctgcgccgtcttctggtggttcggcgtcttcaacgaacactaacagctccatagaagcgaagaactattcggatttagtgagaggtaaccgtatcggaagcggagcaggtggaacggtatacaaagtgattcaccgtccgagttctcgtctatatgcacttaaggtgatatacggtaaccacgaggagactgtgagacgtcagatctgtagagagatcgagattttacgagatgtgaatcatccaaacgttgtgaaatgtcacgagatgtttgatcagaacggtgagatccaggttttgcttgagtttatggataaaggttctttagaaggtgctcatgtgtggaaagagcaacaattagctgatctatctcgtcagattcttagtggtttagcttatctccatagccgtcacatagttcatcgtgatatcaaaccatcgaatcttttgataaactctgctaaaaacgttaagattgctgattttggagttagtaggatcttggctcagactatggatccgtgtaattcatctgttggaaccattgcttatatgagtcctgagaggattaacactgatttgaatcagggaaagtatgatggttatgctggagatatttggagcttaggtgttagcattttggagttttacttggggaggtttcctttccctgtgagtagacaaggtgattgggctagtcttatgtgtgccatttgtatgtctcagcctccagaagctccagcgactgcgtcgccggagtttcggcattttatctcgtgttgcttgcagagagaaccggggaaaaggaggagtgctatgcagctattgcagcatcctttcatattaagagcaagtccgagccagaacaggtctcctcagaatctacatcaactcttgcctcctcctcgtcctctgtcctcgtcttcttctccaaccacatag

SEQ ID NO: 4 - protein, 366 aa, Arabidopsis thaliana
MRPIQSPPGVSVPVKSRPRRRPDLTLPLPQRDVSLAVPLPLPPTSGGSGGSSGSAPSSGGSASSTNTNSSIEAKNYSDLVRGNRIGSGAGGTVYKVIHRPSSRLYALKVIYGNHEETVRRQICREIEILRDVNHPNVVKCHEMFDQNGEIQVLLEFMDKGSLEGAHVWKEQQLADLSRQILSGLAYLHSRHIVHRDIKPSNLLINSAKNVKIADFGVSRILAQTMDPCNSSVGTIAYMSPERINTDLNQGKYDGYAGDIWSLGVSILEFYLGRFPFPVSRQGDWASLMCAICMSQPPEAPATASPEFRHFISCCLQREPGKRRSAMQLLQHPFILRASPSQNRSPQNLHQLLPPPRPLSSSSSPTT

SEQ ID NO: 5 - DNA, 82 bp, Arabidopsis thaliana
cttggctcaggatatggatccgtgtaatgaatctgttggaactccaacagattcattacacggatccatatcctgagccaag

SEQ ID NO: 6 - DNA, 1101 bp, Arabidopsis thaliana
atgagaccgattcaatcgcctccaggagtttccgttccggtgaaaagccgtccccgtcgccgtcctgatcttaccttaccgcttcctcaacgcgatgtttctctcgctgtacctcttcctctcccacctacttccggtggttccggtggctctagtggatctgcgccgtcttctggtggttcggcgtcttcaacgaacactaacagctccatagaagcgaagaactattcggatttagtgagaggtaaccgtatcggaagcggagcaggtggaacggtatacaaagtgattcaccgtccgagttctcgtctatatgcacttaaggtgatatacggtaaccacgaggagactgtgagacgtcagatctgtagagagatcgagattttacgagatgtgaatcatccaaacgttgtgaaatgtcacgagatgtttgatcagaacggtgagatccaggttttgcttgagtttatggataaaggttctttagaaggtgctcatgtgtggaaagagcaacaattagctgatctatctcgtcagattcttagtggtttagcttatctccatagccgtcacatagttcatcgtgatatcaaaccatcgaatcttttgataaactctgctaaaaacgttaagattgctgattttggagttagtaggatcttggctcaggatatggatccgtgtaatgaatctgttggaaccattgcttatatgagtcctgagaggattaacactgatttgaatcagggaaagtatgatggttatgctggagatatttggagcttaggtgttagcattttggagttttacttggggaggtttcctttccctgtgagtagacaaggtgattgggctagtcttatgtgtgccatttgtatgtctcagcctccagaagctccagcgactgcgtcgccggagtttcggcattttatctcgtgttgcttgcagagagaaccggggaaaaggaggagtgctatgcagctattgcagcatcctttcatattaagagcaagtccgagccagaacaggtctcctcagaatctacatcaactcttgcctcctcctcgtcctctgtcctcgtcttcttctccaaccacatag

SEQ ID NO: 7 - protein, 366 aa, Arabidopsis thaliana
MRPIQSPPGVSVPVKSRPRRRPDLTLPLPQRDVSLAVPLPLPPTSGGSGGSSGSAPSSGGSASSTNTNSSIEAKNYSDLVRGNRIGSGAGGTVYKVIHRPSSRLYALKVIYGNHEETVRRQICREIEILRDVNHPNVVKCHEMFDQNGEIQVLLEFMDKGSLEGAHVWKEQQLADLSRQILSGLAYLHSRHIVHRDIKPSNLLINSAKNVKIADFGVSRILAQDMDPCNESVGTIAYMSPERINTDLNQGKYDGYAGDIWSLGVSILEFYLGRFPFPVSRQGDWASLMCAICMSQPPEAPATASPEFRHFISCCLQREPGKRRSAMQLLQHPFILRASPSQNRSPQNLHQLLPPPRPLSSSSSPTT

SEQ ID NO: 8 - DNA, 1047 bp, Arabidopsis thaliana
atgaaaccgattcaatctccttctggagtagcttcacctatgaagaaccgtttacgcaaacgtcctgacctaagcttaccactcccacaccgcgacgtcgctctcgccgtacctctccctctcccacctccttcttcctcttcatccgctccggcgtcttcctccgcgatctcaaccaacatctccgccgctaaaagcttatccgagctagaacgagtgaaccgaatcggaagcggagccggaggaacggtttacaaagtaatccacactccgacgtcacgtcctttcgctctcaaagtgatttacggaaaccacgaagataccgtgagacgtcagatctgtagagagatcgagatcttaagaagtgttgatcatccaaacgttgtgaaatgtcacgatatgtttgatcataacggtgagatccaggttttgcttgagtttatggatcaaggatctcttgaaggagctcatatatggcaagaacaggaattagctgatctctctcgtcagattcttagtggattagcttatcttcatcgtcgtcatatcgttcatcgtgatatcaaaccttcgaatctccttataaactcagctaaaaatgtgaaaattgctgattttggtgtgagtaggatcttggcacaaacaatggatccttgtaattcatctgttggtactattgcttatatgagtcctgagaggattaatactgatttgaatcatggtcgttacgatggttatgctggagatgtttggagtttaggtgttagtatcttggagttttacttggggaggtttccttttgctgtgagtagacaaggtgattgggctagtcttatgtgtgctatttgtatgtctcagccacctgaagctccggctacggcgtctcaggagtttcgtcactttgtttcttgttgtttacagagtgatcctcctaagagatggtcagctcaacagcttttgcagcatcctttcatacttaaagctaccggtggtcctaatctccgtcaaatgttgccgccgcctcgtcctcttccttctgcctcttag

SEQ ID NO: 9 - protein, 348 aa, Arabidopsis thaliana
MKPIQSPSGVASPMKNRLRKRPDLSLPLPHRDVALAVPLPLPPPSSSSSAPASSSAISTNISAAKSLSELERVNRIGSGAGGTVYKVIHTPTSRPFALKVIYGNHEDTVRRQICREIEILRSVDHPNVVKCHDMFDHNGEIQVLLEFMDQGSLEGAHIWQEQELADLSRQILSGLAYLHRRHIVHRDIKPSNLLINSAKNVKIADFGVSRILAQTMDPCNSSVGTIAYMSPERINTDLNHGRYDGYAGDVWSLGVSILEFYLGRFPFAVSRQGDWASLMCAICMSQPPEAPATASQEFRHFVSCCLQSDPPKRWSAQQLLQHPFILKATGGPNLRQMLPPPRPLPSAS

SEQ ID NO: 10 - DNA, 80 bp, Arabidopsis thaliana
cttggcacaagaaatggatccttgtaatgaatctgttggtaccaacagattcattacaaggatccatttcttgtgccaag

SEQ ID NO: 11 - DNA, 1047 bp, Arabidopsis thaliana
atgaaaccgattcaatctccttctggagtagcttcacctatgaagaaccgtttacgcaaacgtcctgacctaagcttaccactcccacaccgcgacgtcgctctcgccgtacctctccctctcccacctccttcttcctcttcatccgctccggcgtcttcctccgcgatctcaaccaacatctccgccgctaaaagcttatccgagctagaacgagtgaaccgaatcggaagcggagccggaggaacggtttacaaagtaatccacactccgacgtcacgtcctttcgctctcaaagtgatttacggaaaccacgaagataccgtgagacgtcagatctgtagagagatcgagatcttaagaagtgttgatcatccaaacgttgtgaaatgtcacgatatgtttgatcataacggtgagatccaggttttgcttgagtttatggatcaaggatctcttgaaggagctcatatatggcaagaacaggaattagctgatctctctcgtcagattcttagtggattagcttatcttcatcgtcgtcatatcgttcatcgtgatatcaaaccttcgaatctccttataaactcagctaaaaatgtgaaaattgctgattttggtgtgagtaggatcttggcacaagaaatggatccttgtaatgaatctgttggtactattgcttatatgagtcctgagaggattaatactgatttgaatcatggtcgttacgatggttatgctggagatgtttggagtttaggtgttagtatcttggagttttacttggggaggtttccttttgctgtgagtagacaaggtgattgggctagtcttatgtgtgctatttgtatgtctcagccacctgaagctccggctacggcgtctcaggagtttcgtcactttgtttcttgttgtttacagagtgatcctcctaagagatggtcagctcaacagcttttgcagcatcctttcatacttaaagctaccggtggtcctaatctccgtcaaatgttgccgccgcctcgtcctcttccttctgcctcttag

SEQ ID NO: 12 - protein, 348 aa, Arabidopsis thaliana
MKPIQSPSGVASPMKNRLRKRPDLSLPLPHRDVALAVPLPLPPPSSSSSAPASSSAISTNISAAKSLSELERVNRIGSGAGGTVYKVIHTPTSRPFALKVIYGNHEDTVRRQICREIEILRSVDHPNVVKCHDMFDHNGEIQVLLEFMDQGSLEGAHIWQEQELADLSRQILSGLAYLHRRHIVHRDIKPSNLLINSAKNVKIADFGVSRILAQEMDPCNESVGTIAYMSPERINTDLNHGRYDGYAGDVWSLGVSILEFYLGRFPFAVSRQGDWASLMCAICMSQPPEAPATASQEFRHFVSCCLQSDPPKRWSAQQLLQHPFILKATGGPNLRQMLPPPRPLPSAS

SEQ ID NO: 13 - protein, 366 aa, Arabidopsis thaliana; identical to SEQ ID NO: 4

SEQ ID NO: 14 - protein, 348 aa, Arabidopsis thaliana; identical to SEQ ID NO: 9

SEQ ID NO: 15 - DNA, 2562 bp, Arabidopsis thaliana
tcgtccatgatggacatatatgtgactgcgtatttacacacaccgcacgtatgcttatttcacacgttagaagaagaattcaaagaagtcggttctatttgtattcttgtgagatcatcaatatgacaatatcgttctattaatatacgtataattcatatgttgtcatggtttcacataccatgtcgacagtcgacgtacgtacaaaagtataaatagtatgaatctaataacagcaccaagattgaagttcatcttctaatcaaaactatcataaagtggtttcaaaatagtgttttttctgatgaaactataactgagttataatcaatccgaaattatataactaattatatttgggaactagataaacgcaaaaacatgagcagtttcttattttttttgtccagatttaaaatttggagtgttaaaatatacggagtgttacacaatgaaaacacaagaagtcaagaacccataagttattttaattaataatattgtatttaaagtgattattaaaaaataatgtaaaaactgattatttgttgacaaaaaacagttagttatagttaaatagtattgatgcatatatatactatctcattattttggtattactcagtactcacatctttaataaagacaaagatagttagtgtataattcaaatcgaactcacagaagtcaataagcgcgtaaaaatacaaaaatatctggcagactttagcaaggtttgttttccaacagaaatggtcatttcagaatcatttacatatccatatatatagctcttaaatggtatatattgggtaatgggtattcgtttaaataattttgttttctgtaaatttcaaatattaatctgatcagtttatccatgtgtgtatatttagtgtattatcatcaatatatgacatagacagactttcaagttggtgcaagaggggatgaaaatttcttcccagttgcaagagtaagctgactagcattttttttttatatataatttatttctcaaatggtttttattattgttttgttgactttaagtttttgccttttatgggactgcaatcacccgtgccaacttttaatctccatcgcctaaaaaagaaagaaaaggctaccattatggaccgaaatatttaagaccataatacaaaattatacgaatattttctgtaacttatatatatgatcatttgacaaagcaaagtcaatcaaataaacttcaaagaaattatgagcttataataagtttgatagtgttaaatataatcaaatcattaaatttagtatttatttcatctcggtttccatttgatagatagataaatgaaagatagcatcgccaataatgaaaaaactttatttgatggcaatactttgttacatcatttctgttttcttaatttcatgtcgaaatattgccatgattgtgttcaacataactagttttgaggtaacaagttaaaaatttgttatattttttgaatatgttattcagttgaaagtcatttagatgtaagtaaaaacaaacataagaagttaacatatcaatattaacacagcgaataatcattattacaaaaaaaagcaaaaaaatagaagaagatattatatattggagaatctttctttagtcttagttgggaagattttgtgctatgggattaaaggtatccatccctattctatgatagaggcgtggggttattggaccaatctatatatattaccacaaggcttaagatgaagtgataatacagtattattaataccctcccaaattatttttaaatatttatcaaaagaagcttacggtatagatcatacttgcagcattattctataagtttatttaatttcagtggctcgttacgtgaacacaaggtaagctaatagacttacgtgccccattaaacacatacataattatacaagtatcatgaaactagtgacaaaacctcgatcaaataaagaaattaccatgacgacaaaagataattaaaaaaaaaactactatatgtcatactcatgcatatgcatgtacaaatgccgctttaaatatttaatttagttaaagcaatgatatttaaattctctctacttcatatatattccaaaagacatattgtcaaattcctttttttagttatatataatcatatattcatattgttatattttcaatatttaatagtaagatggactttcctgaatgttgtgtatgatttataatttgagatattttgtcggagatggatatttgacaagttaatgttactttattaaaattttctaaacatttaggtacgaattgactttttcaaaagtcaacacaataaattttaaaagtttaatgacttaacgggttcacatgggaaacgaaaacaccctaaaccacaaacaatctaatcttatttccttctttatataaaccgctgtttcccaaaaggcttgttctcgtcatatgtacttgtacaccaacccaccaaaagagataaaagaggaaacaaaaactcgaaaagagagagatatatgggtgaggtggcttat

SEQ ID NO: 16 - DNA, 2796 bp, Arabidopsis thaliana
ctgacagtgaacttcattgttcaagcgaggtgagtttcctatttttcttcttctcctttcaattaaaatttcagggtttatgatctctagggtttaggttttattttcttaaactaaaccctaaattctttttcttcttctttttcttgtaatttccagatgcactgcgaacgctagaggaggtgcgatatcgaagaggaagcgttatacaggatcatgggaccaaaatatatcgtatagattcggttcttgttcttctataccttatgagagtagaagtttttctcttcaagagaaaaaaaaaaaaaagaaaaaaaaaaaagaagagtagaaatttctctgtgtttttttttaccaacaagacacaaatgaaactggtccaaaaggagtgtgtataatctctgtggagacataactaatacgttgatgaatttcaagaatacttggattatatagattaaccctgactcccttagatagagatcgaaatcgggtggtgatttctgagaccatacaagttctgatcagattactactaagtaaatcttaattattagactgtttttaatggatttttctgctctaatgataacgtaatgagattttttttttttttttgtatgtttggcttgcagacccgggtaatgagaagtttggtaagagcaaaaggcactaatctcacgtaagaaaacacttttttcatcaaccatgtatataatcatgtcggtttacataaaccgtatcgtctattcaagaatttagttttgtataattataatatttttttcagactacttttcaattaagcatctttttcttggatttttttttcacaaagggagagctttaactttttgcattaacttatatattttaaattatacatgcatgcataccgacttatataaatcatatggtcaatatgagacttttgatttatattatttgtcaactaagcatcttttcagatgaggttcatgcacctttgttagaattatcggaccagaagatcacatcaacgtttaccaaatcaacaaaaaaatccaatccgtccaaaaaatttggaaactgtttgaaagattcgaaatgttggagcaaggatactcagttccaatctctgagcagaatctgatatgactcatctactcataagactttgcgagatagaccggtacaaaaccgtttccaagggttcataatatatggattaatgtgagttattgtggacgttgtggttgtagaagccgcggtagtcgtggaaacactaattagttatctcctgtaagctatttttattttttttcctcacttcctcttcctcgcagctatgtataattttggttggttatctccaaaactttttatggtcaaacaattttgagaaagtaatttggaaagaaaaaaggtttaagaatatctttttatcgtcaagtcctcctccaaattacttccttaaaactgtttgaccataaaaagtcatagcaaggtattagttattctaatttaaattaaacgcctttttatcaacaacaaaaaaaagaagaagatttgatatgttgaaaagtattagggacgcttattagggcagtattcctagttattgcattttctttcggtcattcgaccttaggatcatacctcaatctgtatgactgtattcgccatgtgaattccaatattacatagtgaccaaatttgatatccaactaaaagtcgatctttgatctaaacgaaatgacaactatttggttagtgattgcaggttggaaagatttaccttctagacctgtcttacgaagctagtattctaaagtaatcttcataaaccgaattcagaaacaaaaaaagaaaaggagtccaaaattgtatgatcatacattaatatcagaatagtctcttttgttaaataaatatctgaagaatatatatctctttgattattttgtggatggcaatgaaactaagaatatatattcattgacttagaagtcgacaaaaaaaaataaaaaaattattgacttaattactagttgaccaatatatatattattaaaagaacatattgtatcgttgaaagcggatcatcgggttttaaaagaaaaacacatcgttgaaacttgaaagtgatgactaataaaaagatctaaacgtgtccggtcacctaccaatgtggttttgcaaattattgtcaagtaccttgactatattaaataaaaaaattcaccgtaacacattgatattcaactgattcctaaaaaaatatacaaactattgggagttgtgagattttttatatcagtgttggtctctttacatttgtgatgtggtgttatagcatatatagtaataaactcaaaaggaaattagatgtgttttgaccatttattaaaatgaaccttttcttgtcaaacatttgaaaaatactagtttttttttttggcaacgttgtaaataatagttaaaaatagattttaagtctcgtttttttatgcatatagtttcattcgctttattagactcaaatatacttttaattaaattttgcagagaattaaaggtaatcatttgccaaggaaaaaccatgcaaatatgcaataagtagaaataatgttaatgagagtaagcgttgacatatattacgtcctggtccgaacattcttaaagttgcgtaacactaataaccttagaagatggttggttgactatcaacatcttattgaccaaatgtttttttttttttaattataaaacagttgctcattgctctagcccagagaaagcagctcaattaagtaa

(19) United States
(12) Patent Application Publication
(10) Pub. No.: US 2005/0262584 A1
(43) Pub. Date: Nov. 24, 2005

(54) Master activators of pathogen responsive genes

(76) Inventors: Jen Sheen, Boston, MA (US); Frederick M. Ausubel, Newton, MA (US); Tsuneaki Asai, Isehara (JP); Guillaume Tena, Boston, MA (US)

Correspondence Address: CLARK & ELBING LLP, 101 FEDERAL STREET, BOSTON, MA, 02110, US

(21) Appl. No.: 10/661,426

(22) Filed: Sep. 12, 2003

Related U.S. Application Data
(63) Continuation of application No. PCT/US02/07650, filed on Mar. 12, 2002.
(60) Provisional application No. 60/275,199, filed on Mar. 12, 2001.

(51) Int. Cl. A01H 1/00; C12N 15/82; C12N 9/12

(52) U.S. Cl. 800/279; 435/468; 435/194

(57) ABSTRACT

Disclosed is a complete plant MAP kinase cascade (e.g., MEKK1, MKK4/MKK5, and MPK3/MPK6) and WRKY22/WRKY29 transcription factors that function downstream of the flagellin receptor FLS2, a leucine-rich-repeat (LRR) receptor kinase. Activation of such a MAPK cascade confers resistance to both bacterial and fungal pathogens. Also disclosed are disease-resistant plants expressing one or more members of this MAP kinase signaling cascade. Such members include constitutively active MEKK1 (ΔMEKK1), MKK4 (MKK4a), and MKK5 (MKK5a) or wild-type WRKY29.

### CROSS REFERENCE TO RELATED APPLICATIONS

This application is a continuation of International Application No. PCT[US02/07650, filed Mar. 12, 2002, published in English under PCT article 21(2), currently pending, which claims benefit of U.S. provisional application to 60/275,199, filed Mar. 12, 2001, each of which are hereby incorporated by reference.

### BACKGROUND OF THE INVENTION

The invention relates to plant disease resistance.

Despite recent progress in understanding the genetic control of plant resistance to pathogens, little progress has been reported in the identification and analysis of key regulators of pathogen resistance. Characterization of such genes would allow for the genetic engineering of plants with a variety of desirable traits. The present invention addresses these and other needs.

### SUMMARY OF THE INVENTION

In one aspect, the invention features a method of enhancing resistance to a plant pathogen in a plant, the method including the steps of: (a) providing a plant cell that expresses an isolated nucleic acid molecule (e.g., a DNA molecule) encoding a kinase domain of a MAPKK polypeptide; and (b) regenerating a plant from the plant cell wherein the isolated nucleic acid molecule is expressed in the plant, and wherein the plant has enhanced resistance to a plant pathogen compared to a corresponding untransformed plant. In preferred embodiments, the plant is a dicot (e.g., a crucifer such as Arabidopsis) or a monocot. In other preferred embodiments, the kinase domain is constitutively active. And in still other embodiments, the MAPKK polypeptide is MKK4 or MKK5. Preferably, the MAPKK polypeptide activates a gene involved in a pathogen defense response (for example, PAL1, GST1, WRKY29, or PR1 genes).

In another aspect, the invention features a method of enhancing resistance to a plant pathogen in a plant, the method including the steps of: (a) providing a plant cell that expresses an isolated nucleic acid molecule (e.g., a DNA molecule) encoding a kinase domain of a MAPKKK polypeptide; and (b) regenerating a plant from the plant cell wherein the isolated nucleic acid molecule is expressed in the plant, and wherein the plant has enhanced resistance to a plant pathogen compared to a corresponding untransformed plant. In preferred embodiments, the plant is a dicot (e.g., a crucifer such as Arabidopsis) or a monocot. In preferred embodiments, the kinase domain is constitutively active, for example, a truncated form of an MAPKKK polypeptide. Preferably, the MAPKKK polypeptide is MEKK1 or ANP1. In other preferred embodiments, the MAPKKK polypeptide activates a gene involved in a pathogen defense response (for example, genes such as the PAL1, GST1, WRKY29, or PR1).

In yet another aspect, the invention features a method of enhancing resistance to a plant pathogen in a plant, the method including the steps of: (a) providing a plant cell that expresses an isolated nucleic acid molecule (e.g., a DNA molecule) encoding a polypeptide having substantial identity to a WRKY polypeptide; and (b) regenerating a plant from the plant cell wherein the isolated nucleic acid molecule is expressed in the plant, and wherein the plant has enhanced resistance to a plant pathogen compared to a corresponding untransformed plant. In preferred embodiments, the plant is a dicot (e.g., a crucifer such as Arabidopsis) or a monocot. In preferred embodiments, the WRKY polypeptide (such as a WRKY29 or WRKY22 polypeptide) induces its own gene expression.

In another aspect, the invention features an isolated nucleic acid molecule having a nucleotide sequence for a gene promoter that initiates pathogen-inducible transcription in a plant cell, wherein the nucleotide sequence is selected from the group consisting of: (a) a nucleotide sequence including the sequence set forth in FIGS. 15 or 16; (b) a nucleotide sequence including at least 40 contiguous nucleotides of the sequence set forth in FIGS. 15 or 16; and (c) a nucleotide sequence that has at least about 70% sequence identity to a sequence set forth in (a) or (b).

In another aspect, the invention features a method for expressing a heterologous nucleotide sequence in a plant, the method including transforming a plant cell with a DNA construct including the heterologous nucleotide sequence operably linked to a promoter that is capable of initiating transcription in a plant cell and regenerating a stably transformed plant from the plant cell, wherein the promoter includes a nucleotide sequence selected from the group consisting of: (a) a nucleotide sequence including the sequence set forth in FIGS. 15 or 16; (b) a nucleotide sequence including at least 40 contiguous nucleotides of the sequence set forth in FIGS. 15 or 16; and (c) a nucleotide sequence that has at least about 70% sequence identity to a sequence set forth in (a) or (b).

In general, the kinase domain used in the methods or plants (e.g., transgenic plants or plants that are bred using a transgenic plant) of the invention is generally expressed by itself, as a MAPKKK polypeptide or a MAPKK polypeptide or kinase domain-containing fragment thereof, or as part of a genetically engineered chimeric polypeptide. Useful kinase domains include those that are capable of activating a gene involved in a plant defense response. Exemplary kinase domains include, without limitation, those that are substantially identical to the kinase domains of NPK1 or an ANP (e.g., ANP1, ANP2, or ANP3) or AtMEKK1 or MEKK4 or MEKK5.

Preferably, the methods and plants of the invention specifically utilize the kinase domain of NPK1 or ANP1. In other preferred embodiments, a full-length MAPKKK polypeptide or a kinase domain-containing fragment thereof that is substantially identical to any one of NPK1, ANP1, ANP2, or ANP3 is utilized.

The DNA encoding the constitutively active kinase domain is, in general, constitutively expressed, as is described herein. However, if desired, the kinase domain is inducibly expressed, or such a domain is expressed in a cell-specific, tissue-specific, or organ-specific manner.

In addition, WRKY polypeptides (such as WRKY29 and WRKY22) are expressed to activate plant pathogen defense responses.

Exemplary plants which are useful in the methods of the invention, as well as for generating the plants (or plant cells, plant components, plant tissues, or plant organs) of the invention, include dicots and monocots, such as sugar cane, wheat, rice, maize, sugar beet, barley, manioc, crucifer, mustard, potato, soybean, sorghum, cassava, banana, grape, oats, tomato, millet, coconut, orange, rye, cabbage, apple, grape, eggplant, watermelon, canola, cotton, carrot, garlic, onion, pepper, strawberry, yam, papaya, peanut, onion, legume, bean, pea, mango, and sunflower.

By “polypeptide” is meant any chain of amino acids, regardless of length or post-translational modification (for example, glycosylation or phosphorylation).

By “substantially identical” is meant a polypeptide or nucleic acid exhibiting at least 40%, preferably 50%, more preferably 80%, and most preferably 90%, or even 95% sequence identity to a reference sequence (for example, the amino acid sequences of the kinase domains or full-length MAPKKK polypeptides of NPK1, MEKK1,ANP1, ANP2, or ANP3 or to their respective nucleic acid sequences, a MAPKK polypeptides of MKK4 and MKK5 or to their respective nucleic acid sequences, or to a WRKY polypeptide, such as WRKY29 or WRKY22 or to their respective nucleic acid sequences).

For polypeptides, the length of comparison sequences will generally be at least 16 amino acids, preferably at least 20 amino acids, more preferably at least 25 amino acids, and most preferably 35 amino acids or greater. For nucleic acids, the length of comparison sequences will generally be at least 50 nucleotides, preferably at least 60 nucleotides, more preferably at least 75 nucleotides, and most preferably 1 10 nucleotides or greater.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, FastA, or PILEUP/PRETTYBOX programs). Such software matches identical or similar sequences by assigning degrees of homology to various substitutions, deletions, and/or other modifications. Conservative substitutions typically include substitutions within the following groups: glycine alanine; valine, isoleucine, leucine; aspartic acid, glutamic acid, asparagine, glutamine; serine, threonine; lysine, arginine; and phenylalanine, tyrosine.

By “obtained from” is meant isolated from or having the sequence of a naturally-occurring sequence (e.g., a cDNA, genomic DNA, synthetic DNA, or combination thereof).

By “isolated nucleic acid molecule” is meant a nucleic acid molecule (e.g., a DNA molecule) that, is free of the genes which, in the naturally-occurring genome of the organism from which the nucleic acid of the invention is derived or obtained from, flank the gene. The term therefore includes, for example, a gene or fragment thereof that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. It also includes a nucleic acid which is part of a hybrid gene encoding additional polypeptide sequence.

By “transformed cell” is meant a cell into which (or into an ancestor of which) has been introduced, by means of recombinant DNA techniques, a DNA molecule encoding (as used herein) a MAPKKK kinase domain (e.g., NPK1, MEKK1, ANP1, ANP2, or ANP3); a MAPKK polypeptide (e.g., MKK4 and MKK5); or a WRKY polypeptide (e.g., WRKY29 or WRKY22).

By “reporter gene” is meant a gene whose expression may be assayed; such genes include, without limitation, β-glucuronidase (GUS), luciferase (LUC), chloramphenicol transacetylase (CAT), green fluorescent protein (GFP), and β-galactosidase.

By “a promoter functional in a plant cell” is meant any minimal sequence sufficient to direct transcription in a plant cell. Included in the invention are promoter elements that are sufficient to render promoter-dependent gene expression controllable for cell-, tissue-, or organ-specific gene expression, or elements that are inducible by external signals or agents (for example, light-, pathogen-, wound-, stress-, or hormone-inducible elements, or chemical inducers) or elements that are capable of cycling gene transcription; such elements may be located in the 5′ or 3′ regions of the native gene or engineered into a transgene construct.

By “operably linked” is meant that a gene and a regulatory sequence(s) are connected in such a way as to permit gene expression when the appropriate molecules (for example, transcriptional activator proteins) are bound to the regulatory sequence(s).

By “plant cell” is meant any self-propagating cell bounded by a semi-permeable membrane and containing a plastid. Such a cell also requires a cell wall if further propagation is desired. Plant cell, as used herein, includes, without limitation, algae, cyanobacteria, seeds, suspension cultures, embryos, meristematic regions, callus tissue, leaves, roots, shoots, gametophytes, sporophytes, pollen, and microspores.

By “plant component” is meant a part, segment, or organ obtained from an intact plant or plant cell. Exemplary plant components include, without limitation, somatic embryos, leaves, stems, roots, flowers, tendrils, fruits, scions, and rootstocks.

By “crucifer” is meant any plant that is classified within the Cruciferae family. The Cruciferae include many agricultural crops, including, without limitation, rape (for example, Brassica campestris and Brassica napus), broccoli, cabbage, brussel sprouts, radish, kale, Chinese kale, kohlrabi, cauliflower, turnip, rutabaga, mustard, horseradish, and Arabidopsis.

By “transgene” is meant any piece of DNA that is inserted by artifice into a cell, and becomes part of the genome of the organism that develops from that cell. Such a transgene may include a gene which is partly or entirely heterologous (i.e., foreign) to the transgenic organism, or may represent a gene homologous to an endogenous gene of the organism.

By “transgenic” is meant any cell that includes a nucleic acid sequence (e.g., a recombinant DNA sequence) that is inserted by artifice into a cell and becomes part of the genome of the organism that develops from that cell. As used herein, the transgenic organisms are generally transgenic plants and the DNA (transgene) is inserted by artifice into the nuclear or plastidic genome.

By “pathogen” is meant an organism whose infection of viable plant tissue elicits a disease response in the plant tissue. Such pathogens include, without limitation, bacteria, mycoplasmas, fungi, oomycetes, insects, nematodes, viruses, and viroids. Examples of plant diseases caused by these pathogens are described in Chapters 11-16 of Agrios, Plant Pathology, 3rd ed., Academic Press, Inc., New York, 1988.

Examples of bacterial pathogens include, without limitation, Erwinia (for example, E. carotovora), Pseudomonas (for example, P. syringae), and Xanthomonas (for example, X. campepestris and X. oryzae).

Examples of fungal or fungal-like disease-causing pathogens include, without limitation, Alternaria (for example, A. brassicola and A. solani), Ascochyta (for example, A. pisi), Botrytis (for example, B. cinerea), Cercospora (for example, C. kikuchii and C. zaea-maydis), Colletotrichum sp. (for example, C. lindemuthianum), Diplodia (for example, D. maydis), Erysiphe (for example, E. graminis fsp. graminis and E. graminis f.sp. hordei), Fusarium (for example, F. nivale, F. oxysporum, F. graminearum, F. solani, F. monilforme, and F. roseum), Gaeumanomyces (for example, G. graminis f.sp. tritici), Helminthosporium (for example, H. turcicum, H. carbonum, and H. maydis), Macrophomina (for example, M. phaseolina and Maganaporthe grisea), Nectria (for example, N. heamatocacca), Peronospora (for example, P. manshurica, P. tabacina), Phoma (for example, P. betae), Phymatotrichum (for example, P. omnivorum), Phytophthora (for example, P. cinnamomi, P. cactorum, P. phaseoli, P. parasitica, P. citrophthora, P. megasperma f.sp. sojae, and P. infestans), Plasmopara (for example, P. viticola), Podosphaera (for example, P. leucotricha), Puccinia (for example, P. sorghi, P. striiformis, P. graminis f.sp. tritici, P. asparagi, P. recondita, and P. arachidis), Puthium (for example, P. aphanidermatum), Pyrenophora (for example, P. tritici-repentens), Pyricularia (for example, P. oryzea), Pythium (for example, P. ultimum), Rhizoctonia (for example, R. solani and R. cerealis), Scerotium (for example, S. rolfsii), Sclerotinia (for example, S. sclerotiorum), Septoria (for example, S. lycopersici, S. glycines, S. nodorum and S. tritici), Thielaviopsis (for example, T. basicola), Uncinula (for example, U. necator), Venturia (for example, V. inaequalis), and Verticillium (for example, V. dahliae and V. albo-atrum).

Examples of pathogenic nematodes include, without limitation, root-knot nematodes (for example, Meloidogyne sp. such as M. incognita, M. arenaria, M. chitwoodi, M. hapla, M. javanica, M. graminocola, M. microtyla, M. graminis, and M. naasi), cyst nematodes (for example, Heterodera sp. such as H. schachtii, H. glycines, H. sacchari, H. oryzae, H. avenae, H. cajani, H. elachista, H. goettingiana, H. graminis, H. mediterranea, H. mothi, H. sorghi, and H. zeae, or, for example, Globodera sp. such as G. rostochiensis and G. pallida), root-attacking nematodes (for example, Rotylenchulus reniformis, Tylenchuylus semipenetrans, Pratylenchus brachyurus, Radopholus citrophilus, Radopholus similis, Xiphinema americanum, Xiphinema rivesi, Paratrichodorus minor, Heterorhabditis heliothidis, and Bursaphelenchus xylophilus), and above-ground nematodes (for example, Anguina funesta, Anguina tritici, Ditylenchus dipsaci, Ditylenchus myceliphagus, and Aphenlenchoides besseyi).

Examples of viral pathogens include, without limitation, tobacco mosaic virus (TMV), tobacco necrosis virus (TNV), potato leaf roll virus, potato virus X, potato virus Y, tomato spotted wilt virus, and tomato ring spot virus.

By “enhanced resistance to a plant pathogen” is meant a level of resistance to a disease-causing pathogen in a non-naturally occurring plant (or cell or seed thereof) which is greater than the level of resistance in a control plant (for example, a non-transgenic plant or wild-type). In preferred embodiments, the level of resistance in a non-naturally occurring transgenic plant of the invention is at least 5% to 20% (and preferably 30% or 40%) greater than the resistance exhibited by a control plant. In other preferred embodiments, the level of resistance to a disease-causing pathogen is 50% greater, 60% greater, and more preferably even 75% or 90% greater than a control plant; with up to 100% or greater above the level of resistance as compared to a control plant being most preferred. The level of resistance is measured using conventional methods. For example, the level of resistance to a pathogen may be determined by comparing physical features and characteristics (for example, plant height and weight) or by comparing disease symptoms (for example, delayed lesion development, reduced lesion size, leaf wilting and curling, water-soaked spots, amount of pathogen growth, and discoloration of cells) of the non-naturally occurring plant (e.g., a transgenic plant).

By “detectably-labeled” is meant any direct or indirect means for marking and identifying the presence of a molecule, for example, an oligonucleotide probe or primer, a gene or fragment thereof, or a cDNA molecule or a fragment thereof. Methods for detectably-labeling a molecule are well known in the art and include, without limitation, radioactive labeling (for example, with an isotope such as ³²P or ³⁵S) and nonradioactive labeling (for example, fluorescence of chemiluminescent labeling, for example, fluorescein labeling).

The invention provides for increased production efficiency, as well as for improvements in quality and yield of crop plants and ornamentals. Thus, the invention contributes to the production of high quality and high yield agricultural products; for example, fruits, ornamentals, vegetables, cereals, and field crops. Genetically-improved seeds and other plant products that are produced using plants expressing the genes and methods described herein also render farming possible in areas previously unsuitable for agricultural production. The invention further provides a means for mediating the expression of pathogen defense response genes (e.g., GST1, PAL, WRKY29, and PR1) that enable a plant to resist its pathogens. For example, transgenic plants constitutively expressing a kinase domain of a MAPKKK or MAPKK, or a WRKY polypeptide are capable of turning on a plant's pathogen defense transduction pathway by activating the expression of plant pathogen defense regulatory pathways.

As discussed above, MAPK activation is responsible for providing plants with the ability to protect themselves against pathogens have been identified. Accordingly, the invention provides a number of important advances and advantages for the protection of plants against their pathogens. For example, expression of such genes in planta, as described herein, facilitates an effective and economical means for in-plant protection against plant pathogens. Such protection against pathogens reduces or minimizes the need for traditional chemical practices (for example, application of fungicides, bactericides, nematicides, insecticides, or viricides) that are typically used by farmers for controlling the spread of plant pathogens and providing protection against disease-causing pathogens.

The invention further provides a means for mediating the expression of pathogenesis-related proteins, for example, GST, that confers resistance to plant pathogens. For example, transgenic plants constitutively producing an MAPK cascade gene product (e.g., a MAPKKK, MAPKK, or WRKY polypeptide) are capable of activating PR gene expression, which in turn confers resistance to plant pathogens. Collective PR gene expression that is mediated by expression of a MAPK cascade polypeptide (e.g., a MAPKKK, MAPKK, or WRKY polypeptide) obviates the need to express individual resistance genes as a means to promote plant defense mechanisms.

Moreover, constitutively active derivatives of MAPKKKs (e.g., MEKK1 and ANP1) and MAPKKs (e.g., MKK4 and MKK5) can activate the PAL1, GST1, and WRKY29, and PR1 gene promoters. Thus, MEKK1, ANP1, MKK4, and MKK5 (and their orthologs in other plants) activate the same MAPK cascades. These PKs are therefore reasonable targets for transgenic plant manipulation to generate agronomically valuable traits, such as broad-spectrum pathogen resistance. The PAL1 gene encodes phenylalanine ammonia lyase that functions at a universal key step for the biosynthesis of salicyclic acid and phytoalexins (anti-pathogen hormones and chemicals). The expression of GST1 (glutathione S-transferase) is important for the protection and detoxification of cells during pathogen infection. The activation of the WRKY29 (a transcription activator and DNA binding protein for the W box) provides a positive feedback loop for the regulation of PAL1 and GST1 genes. The PR1 protein has an important anti-pathogen activity.

Other features and advantages of the invention will be apparent from the following description of the preferred embodiments thereof, and from the claims.

### BRIEF DESCRIPTION OF THE DRAWINGS

The drawings will first be described.

FIG. 1 shows a MAPK signaling pathway leading to WRKY29.

FIG. 2 shows a schematic diagram of the Arabidopsis WRKY29 polypeptide, including the WRKY domain and its W box target sequence.

FIG. 3 shows that Flg22 induces WRKY29 in Arabidopsis protoplasts.

FIG. 4 shows the MKK4 cDNA sequence and its predicted amino acid sequence.

FIG. 5 shows the strategy used to render MKK4 constitutively active.

FIG. 6 shows the MKK5 cDNA sequence and its predicted amino acid sequence.

FIG. 7 shows the strategy used to render MKK5 constitutively active.

FIG. 8 shows the alignment of MKK4 and MKK5 polypeptides.

FIG. 9 shows that constitutively active AtMEKK1 induces WRKY29.

FIG. 10 shows that constitutively active AtMKK4 induces WRKY29.

FIG. 11 shows that WRKY29 induces its own promoter.

FIG. 12 shows that WRKY29 regulates early defense genes. Pal1 refers to phenylalanine ammonia lyase; FRK1 refers to the flg22 induced receptor-like kinase; GST1 refers to glutathione S-transferase 1; and GST6 refers to glutathione S-transferase 6.

FIG. 13 shows a schematic diagram of the Agrobacterium-mediated transient transfection protocol.

FIG. 14 shows that transient expression of WRKY29 reduces susceptibility to Pseudomonas syringae.

FIG. 15 shows the pathogen-inducible WRKY29 gene promoter fragment.

FIG. 16 shows the pathogen-inducible WRKY22 gene promoter fragment.

FIGS. 17A-D show the early defense gene activation by flg22. FIG. 17A shows a RT-PCR analysis. Protoplasts were preincubated for one hour with (+) or without (−)10 μM CHX, and then treated without or with 100 nM flg22. FIG. 17B shows the involvement of MAPK. Transfected protoplasts were preincubated for one hour with 1 μM staurosporine (ST) or with 1 μM U0126 before induction by 100 nM flg22 for four hours. FIG. 17C shows the FLS2 requirement. Protoplasts isolated from wild-type (F) and fls2-24 mutant (f) leaves were transfected with an effector plasmid expressing GFP (−) or FLS2. Transfected protoplasts were incubated for nine hours first before incubation with or without 100 nM flg22 for four hours. FIG. 17D shows MKP1 suppression. Protoplasts were transfected with an effector plasmid expressing GFP (−) or MKP1.

FIGS. 18A-D show that Flg22 activates MPK3 and MPK6 through FLS2. FIG. 18A shows that Flg22 activates endogenous MBP kinases. Protoplasts were treated with 1 μM flg22 (+) or water (−). FIG. 18B shows that Flg22 activates MPK3 and MPK6. MAPK activation with or without 100 nM flg22 for 10 minutes (top). Expression of each MAPK (2, 3, 5, 6, 7 or 9) protein was verified by [³⁵S]methionine labeling and immunoprecipitation (bottom). FIG. 18C shows that Flg22 activation of MPK3 and MPK6 requires FLS2. AN HA-tagged MAPK (3 or 6) was coexpressed with a wild-type (FLS2) or kinase-inactive (FLS2 Km) flagellin receptor with a HA tag in fls2-24 protoplasts. MAPK activities (top) and FLS2 (middle) and MAPK (bottom) protein levels are shown. Control experiments were performed with wild-type (Wt) protoplasts. FIG. 18D shows that MKP1 abolishes flg22 activation of MPK3 and MPK6. MAPK activity (top), and MAPK (middle), and MKP1 (bottom) protein levels are shown.

FIGS. 19A-E show the MKK4 and MKK5 activation of MPK3 and MPK6 and the flg22-inducible early defense genes. FIG. 19A shows that MKK5 activates MPK3 and MPK6. Protoplasts were transfected with plasmids expressing MAPK (2, 3, 5, 6, 7 or 9) and wild-type MKK5 (w) or a constitutively active MKK5 mutant (a). MAPK activity (top) and MAPK (middle), and MAPKK protein levels are shown (bottom). Asterisk indicates a background band. FIG. 19B shows that MKK4 and MKK5 are redundant for MPK3 and MPK6 activation. Protoplasts were transfected with plasmids expressing MAPK (3 or 6) and one of four MYC-tagged and constitutively active MAPKK mutants (1, 2, 4, or 5) or a control plasmid (−). MAPK activity (top), MAPK (middle) and MAPKK (bottom) protein levels are shown. FIG. 19C shows that MKK4a and MKK5a activate WRKY29 and FRK1 promoter activity. Protoplasts were expressing GFP (−) or a constitutively active MKK (1, 2, 4, or 5). FIG. 19D shows that dominant-negative mutants of MKK4 and MKK5 partially inhibit flg22 activation of the WRKY29 and FRK1 promoters. Protoplasts were expressing GFP (−) or a kinase-inactive mutant of MKK4 or MKK5 (4 or 5). FIG. 19E shows that MKK4 and MKK5 act downstream of FLS2. Wild-type (FLS2) and fls2-24 (fls2) protoplasts were expressing GFP (−) or a constitutively active mutant of MKKs.

FIGS. 20A-D show that MEKK1 initiates the flg22 MAPK cascade. FIG. 20A shows that constitutively active MEKK1 activates MKK5. Protoplasts were expressing wild-type MKK5 or a control plasmid (−), and one of four HA-tagged constitutively active MAPKKKs (kinase domain) or a control plasmid (−). MKK5 activity based on GST-MPK6Km as a substrate (top) and MAPKKK (middle) and MAPKK (bottom) protein levels are shown. FIG. 20B shows that constitutively active MEKK1 induces the WRKY29 and FRK1 promoters. Protoplasts were expressing GFP (−) or constitutively active MEKK1 (M) or CTR1 (C). FIG. 20C shows that MEKK1 acts downstream of FLS2. Protoplasts were isolated from wild-type (FLS2) and mutant (fls2) leaves. FIG. 20D shows that dominant-negative mutant of MEKK1 inhibits flg22 activation of the WRKY29 and FRK1 promoters. Protoplasts were expressing a kinase inactive mutant of the full-length MEKK1 (MEKK1 in).

FIGS. 21A-F show that the flg22 MAPK cascade and WRKY29 are important for Arabidopsis defense signaling. FIG. 21A shows that WRKY29-GFP is localized in the nucleus. Visualization of protoplasts expressing WRKY29-GFP (top), a red-fluorescent nuclear marker (middle), and super-imposition (bottom), in the absence of flg22. FIG. 21B shows that WRKY22 and WRKY29 activate early defense genes. Protoplasts were expressing GFP (−), WRKY29 (“29”), WRKY22 (“22”) or WRKY42 (“42”). FIG. 21C shows that WRKY29 acts downstream of FLS2. Protoplasts were solated from wild-type (FLS2) and mutant (fls2) leaves. FIG. 21D shows that Arabidopsis leaves expressing ΔMEKK1, MKK4a, MKK5a, or WRKY29 exhibited reduced disease symptoms after P. syringae (Ps) infection. The control leaves were infiltrated with 10 mM MgSO₄ (Mg). FIG. 21E shows Arabidopsis leaves expressing ΔMEKK1, MKK4a, or WRKY29 exhibit reduced disease symptoms after B. cinerea infection. The right half of each leaf was infected with B. cinerea. FIG. 21F shows the early stage of B. cinerea development was inhibited on leaves expressing MKK4a. On MKK1a-expressing leaves (MKK1a), germinated spores (s) of B. cinerea formed superficial hyphae (h) and branched appressoria (a) two days after infection. The fungal spores (s) formed only germ tubes (gt) on MKK4a-expressing leaves (MKK4a). Bars: 50 μM.

FIG. 22 shows a model of innate immune signaling activated by LRR receptors in Arabidopsis, mammals, and Drosophila. A putative repressor (R) could control WRKY22 and WRKY29 activity since their overexpression bypasses the requirement of elicitors. The conserved signaling pathways for innate immune responses in animals are summarized based on recent reviews on mammals and Drosophila.

### DETAILED DESCRIPTION

MAPK cascades are, in general, conserved in eukaryotes. Genome sequence comparisons and molecular analysis of signal transduction pathways have revealed the existence of conserved MAPK signaling cascades in eukaryotes that connect environmental and developmental signals to the activation of a variety of species-specific downstream genes. Extensive studies have revealed the functions of five yeast MAPKs in stress, pheromone, and nutrient responses. Based mostly on analysis in cellular systems, four distinct classes of animal MAPKs have been shown to participate in signaling pathways triggered by growth regulators, neurotransmitters, cytokines, and diverse stresses. It appears likely that additional MAPK signaling pathways will be identified. BLAST searches of DNA databases that have been conducted using a conserved signature motif that covers the phosphorylation site TXY of MAPKs identified relatively large families of putative MAPK genes in yeast (6 genes), C. elegans (17 genes), mouse (17 genes), human (27 genes), and Arabidopsis (18 genes). In the case of Arabidopsis, there are at least 7 putative MAPKK genes and 29 MAPKKK genes. Homologs of many of these genes can be found in other plant species. However, the functions of most of these putative MAPK cascade components have not been determined. As is detailed herein, several of these genes are actively involved in regulating the expression of pathogen defense mechanisms.

For cell signaling, down regulation of the MAPK activity is just as important as its up regulation. Inactivation of MAPK can be achieved through dephosphorylation of crucial protein tyrosine and threonine residues by dual-specificity MAPK phosphatases (MKPs), protein tyrosine phosphatases (PTPs), and serine/threonine protein phosphatases PP2C. Homologs of MKP, PTP, and PP2C genes have been identified in yeast, C. elegans, Arabidopsis, and mammals. Depending on their tissue specificity, mode of induction, subcellular localization, and substrate preferences, each PP might participate in the repression of a specific MAPK activity. Signals that activate MAPKs can often activate the transcription of MKPs or PTPs, that might be important for the negative feedback regulation of the MAPK pathways. The roles of most plant MKPs, PTPs, and PP2Cs in MAPK cascades are unknown.

As is detailed below, an Arabidopsis protoplast transient expression assay and four reporter genes have been used to identify protein (PKs) that can activate the early pathogen response genes, GST1, PAL1, and WRKY29, as well as a late response gene, PR1. Constitutively active derivatives of two MAPKKKs (MEKK1 and ANP1) and two MAPKKs (MEK4 and MEK5) activated the PA11, GST1, WRKY29 and PR1 gene promoters. Accordingly, MEKK1, ANP1, MEK4 and MEK5 activate the same MAPK cascades. These PKs are therefore reasonable targets for transgenic plant manipulation to generate agronomically valuable traits, such as broad-spectrum pathogen resistance.

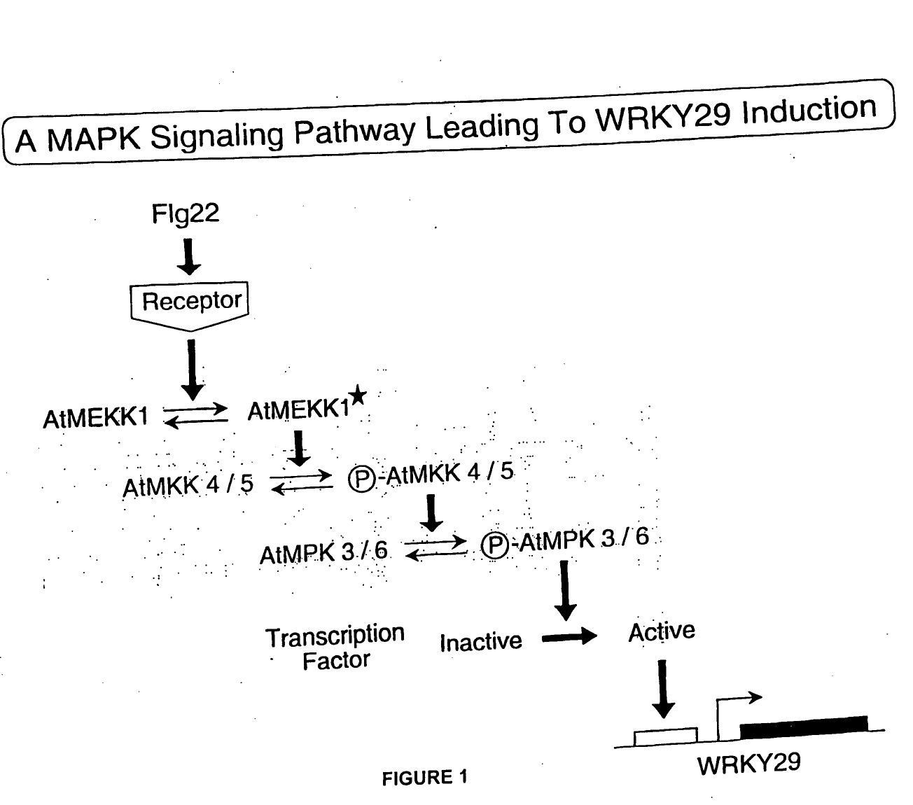

In particular, one MAPK cascade involving Flg22 peptide-mediated defense responses has been examined (FIG. 1). The overall strategy involved the identification and cloning of several Arabidopsis genes encoding MAPKs, MAPKKs, MAPKKKs, and WRKYs. Transient expression of epitope-tagged MAPKs, MAPKKs, and MAPKKKs were then used to determine which MAPK cascades respond to particular signals in Arabidopsis and maize protoplasts. Engineered MAPKKKs and MAPKKs designed to be constitutively active or act as dominant negative inhibitors, as well as WRKYs, were used to identify specific reporter genes associated with particular signaling pathways. Using this system, we have identified a complete plant MAPK cascade and WRKY transcription factors acting downstream of the flagellin receptor FLS2. The data presented below also indicate that this signaling pathway functions in response to both bacterial and fungal pathogens and could therefore be engineered to enhance the disease resistance of crop plants to a wide range of pathogens.

MAPK Activation in the Plant Defense Response

In response to pathogenic microorganisms, plants induce a variety of cellular reactions. Among the reactions are the induction of rapid programmed cell death (PCD), known as the hypersensitive response (HR), and the transcriptional activation of many defense-related genes, including the early GST1 (glutathione S-transferase) and PAL1 (phenylalanine ammonia-lyase) genes, as well as the later pathogenesis-related (PR) genes. Activation of these defense responses is often governed by a specific “gene-for-gene” interaction between a plant resistance (R) gene and a pathogen avirulence (avr) gene. Despite significant insights into R gene structure as putative receptors, little is known about the signal transduction events following R-Avr interactions. The finding that two R gene products, tomato PTO and rice XA-21, contain a protein kinase domain suggest that protein phosphorylation might play a central role in R gene-dependent signal transduction. More recent studies have provided compelling evidence that MAPKs are likely involved in this signaling event. In tobacco, the N gene confers resistance to tobacco mosaic virus (TMV). It was demonstrated that two previously identified MAPKs, a SA-induced protein kinase (SIPK) and a wound-induced protein kinase (WIPK), were activated during infection by TMV. Importantly, WIPK activation by TMV depended on N since the activation was not detected in tobacco plants lacking the N gene. Similarly, treatment of tobacco suspension cells expressing the tomato R gene, Cf-9, with the corresponding avr gene product, Avr9, resulted in rapid activation of both tobacco SIPK and WIPK in a strictly gene-for-gene manner.

Involvement of MAPKs in plant defense signaling has also been demonstrated through the analysis of plant responses after non-specific elicitation with bacterial or fungal oligosaccharides, proteins, or peptides. Parsley cells recognize a fungal pathogen Phytophthora sojae through a plasma membrane receptor. A pathogen-derived oligopeptide elicitor binds to this receptor and thereby stimulates a multi-component defense responses. It was demonstrated that the elicitor activates a MAPK that is similar to tobacco WIPK. A regulatory function of a MAPK in defense gene induction has also been suggested in tobacco suspension cells, in which three fungal elicitors rapidly activate the SIPK.

Another important virulence factor for bacteria that are pathogenic to animals and plants is the flagellum. It has been demonstrated that plants have a highly sensitive chemoperception system for eubacterial flagellins, specifically targeted to the most highly conserved domain within its N terminus. Synthesis of this peptide (22 amino acids) creates a highly active elicitor called Flg22. In Arabidopsis seedlings, Flg22 elicits callose deposition, induction of PR genes, and growth inhibition. Synthetic peptides, carrying the corresponding flagellin sequences of bacteria that coexist intimately with plants such as Rhizobium meliloti and Agrobacterium tumefaciens, fail to induce any defense responses. It was recently reported that, in Arabidopsis, one of the loci conferring Flg22 sensitivity encodes a putative transmembrane protein kinase with leucine-rich repeats, resembling the rice resistance gene XA-21. As is demonstrated herein, the MAPK pathway is involved in Flg22 signaling. There now follow particular examples of the invention. These examples are provided for the purpose of illustrating the invention, and should not be construed as limiting.

### EXAMPLE 1

### Cellular Systems for Functional Analysis of Plant Defense Gene Regulatory Pathways

Two protoplast transient expression systems, such as the maize (Sheen, 1999) or Arabidopsis (Kovtun et al, Proc. Natl. Acad. Sci., USA 97:2940-5, 2000) leaf protoplast systems, are useful for examining regulation of plant defense mechanisms. Technical advances in these two systems, including high transformation efficiency by electroporation (up to 75%) or by PEG fusion (up to 80%), specificity of reporter gene regulation, the use of improved green-fluorescent protein (GFP) as a vital and visual reporter (Chiu et al., Curr. Biol. 6, 325-30, 1996), PK and PP activity assays (Kovtun et al., Nature 395:716-720, 1998; Sheen, Proc. Natl. Acad Sci., USA 95:975-980,1998; Kovtun et al., Proc. Natl. Acad. Sci., USA 97:2940-5, 2000), and expression of putative signaling molecules with epitope- or GFP-tag and their detection by immunoprecipitation and confocal microscopy, make them especially attractive to examine MAPK cascades and the plant defense gene regulatory pathways.

Because MAPK cascades are regulators of pathogen defense the manipulation of such MAPK cascades in plants provides new tools for crop improvement in disease resistance. Furthermore, the high degree of conservation of MAPK signaling pathways facilitates the transfer of knowledge learned in the Arabidopsis system to agricultural species.

### EXAMPLE 2

### Flg22-induced Responses in Arabidopsis Protoplasts

Activation or repression of the transcription of specific reporter genes can be used to monitor activation of particular signaling pathways. For example, Flg22 has been found to induce the expression of the GST1, PAL1, and PAL2 promoters. MAPK activation was demonstrated in Arabidopsis protoplasts isolated from 4-week-old leaves. Here Flg22 or distilled water was added to the protoplasts and samples were assayed at different times to determine relative promoter activity. Defense gene promoters fused to a luciferase reporter gene were introduced into Arabidopsis protoplasts. The protoplasts were incubated for 16 hours in the presence of Flg22 and/or staurosporine, and luciferase activities were assayed. The number of viable cells in each sample was also determined at the end of the incubation by Evans blue staining and promoter activity was represented as a luciferase activity per viable cell. In the Flg22 experiment, the induction of all the promoters was effectively suppressed by staurosporine, a protein kinase inhibitor. However, the GST6 promoter was not induced by Flg22.

### EXAMPLE 3

### Flg22 induces WRKY29 in Arabidopsis Protoplasts and a MAPK Signal Cascade

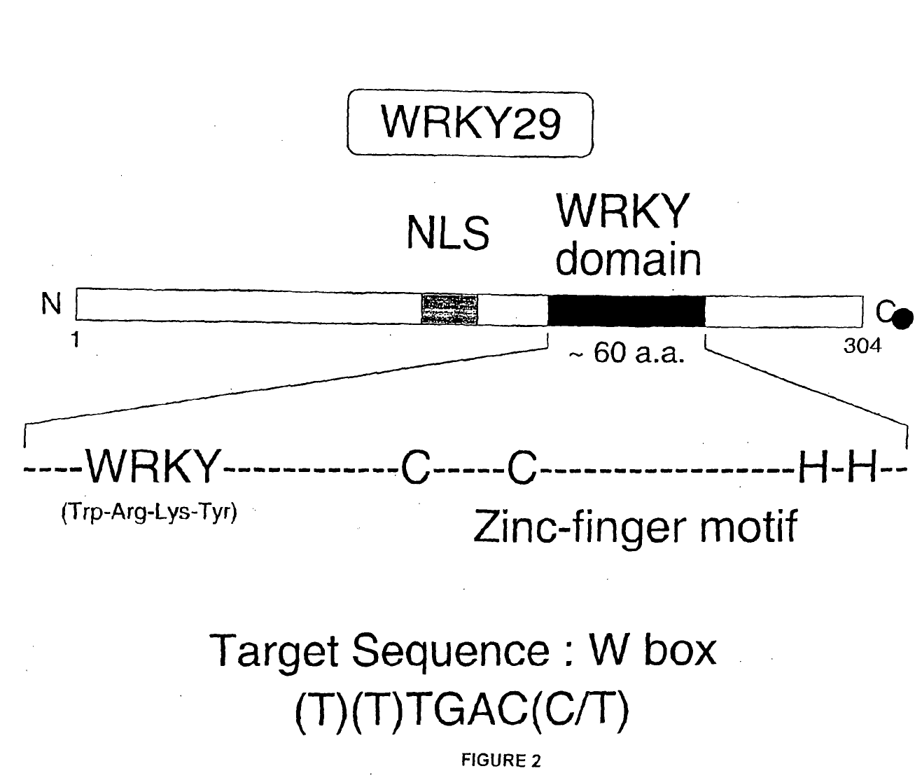

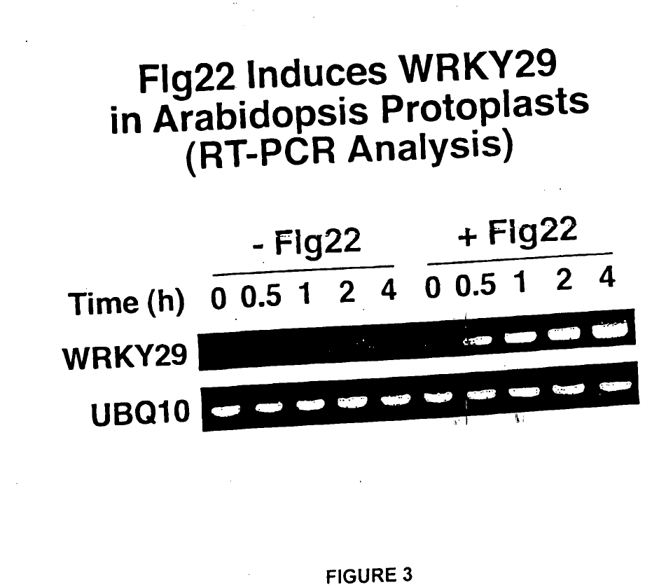

Arabidopsis protoplasts treated Flg22 were evaluated for the expression of WRKY29 using standard reverse-transcription polymerase chain reaction analysis (RT-PCR). As shown in FIG. 3, Flg22 was found to induce WRKY29. The structure of WRKY29 is shown in FIG. 2. To determine whether, MAP kinase signaling is involved in WRKY29 induction, the induction of WRKY29 was monitored in the presence of U0126, a MAPKK inhibitor. RT-PCR analysis of WRKY29 induction showed that U0126 suppressed WRKY29 induction. Furthermore, Flg22 was found to activate MBP kinases. Further demonstrating the involvement of a MAPK cascade, mouse MAPK phosphatase l was found to suppress WRKY induction by Flg22.

### EXAMPLE 4

### Constitutively Active ANP1 and MEKK1 Activate PAL1, GST1, and WRKY29

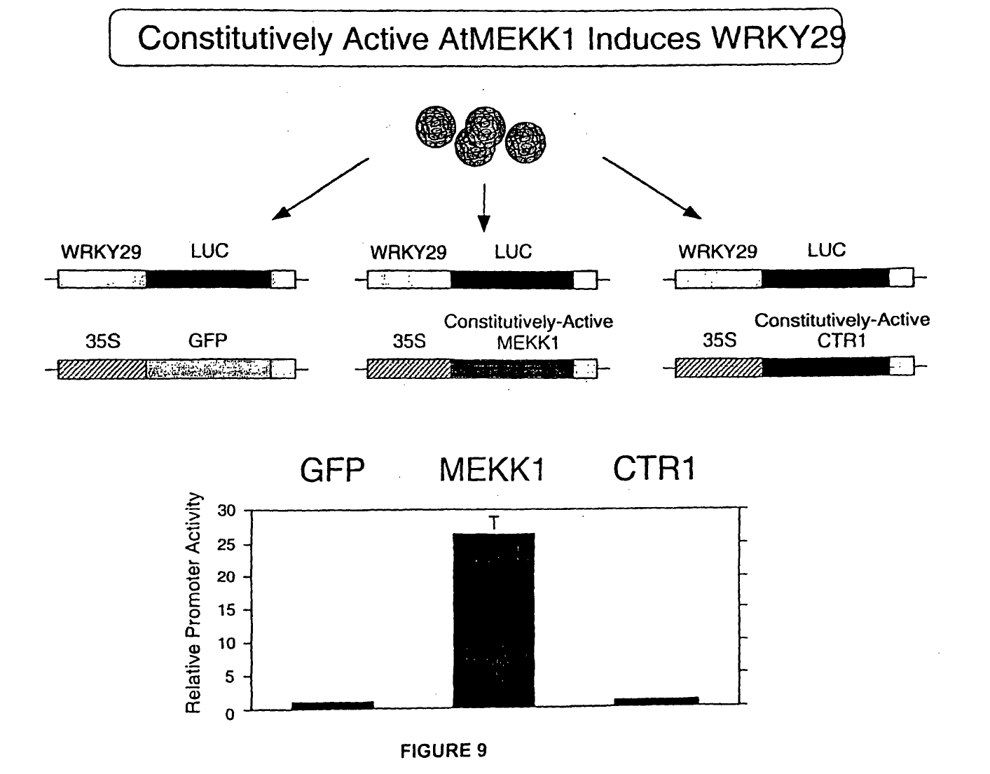

To determine whether ANP1 and MEKK1 were involved in activating plant defense response genes, the effect of a constitutively active ANP1 and MEKK1 on the activity of the PAL1 promoter was examined. These polypeptides were rendered constitutively active according to standard methods known in the art. It has been shown that MAPKKKs consist of a well-conserved kinase domain and putative regulatory domains. Truncated or naturally occurring MAPKKKs carrying only the kinase domain have been shown to have constitutive kinase activity. The expression of the constitutively active ANP1 and MEKK1 was found to activate PAL1 gene expression. In addition, constitutively active ANP1 and MEKK1 was found to activate the GST1 and WRKY29 gene promoters (FIG. 9).

### EXAMPLE 5

### Constitutively Active AtMKK4 and AtMKK5 Activate PAL1 and WRKY29

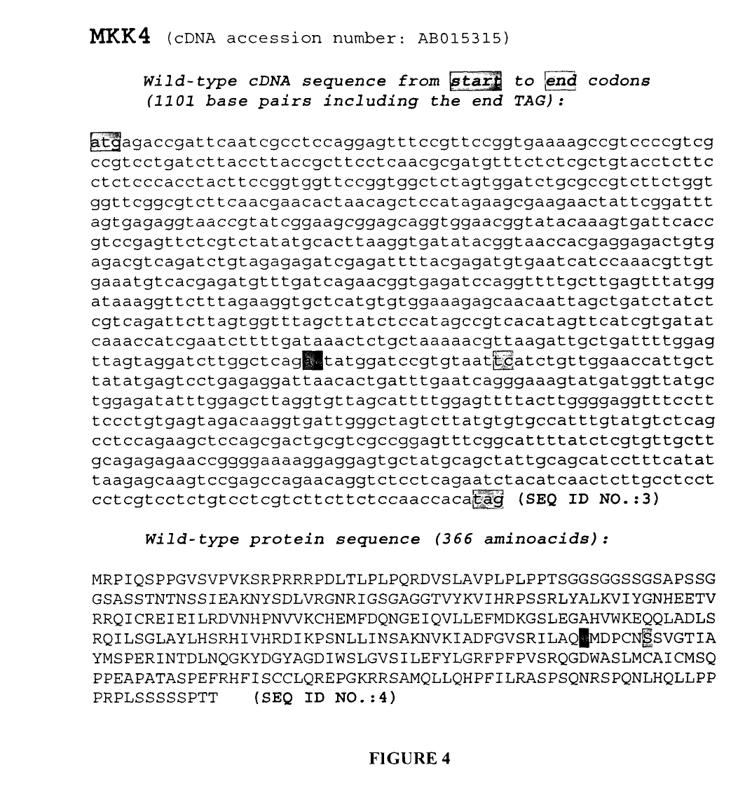

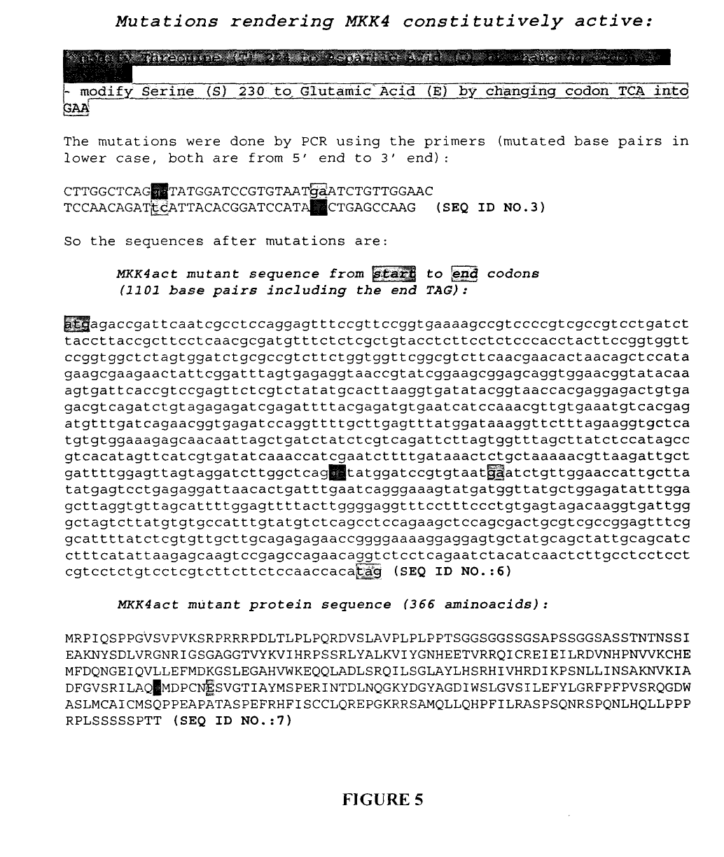

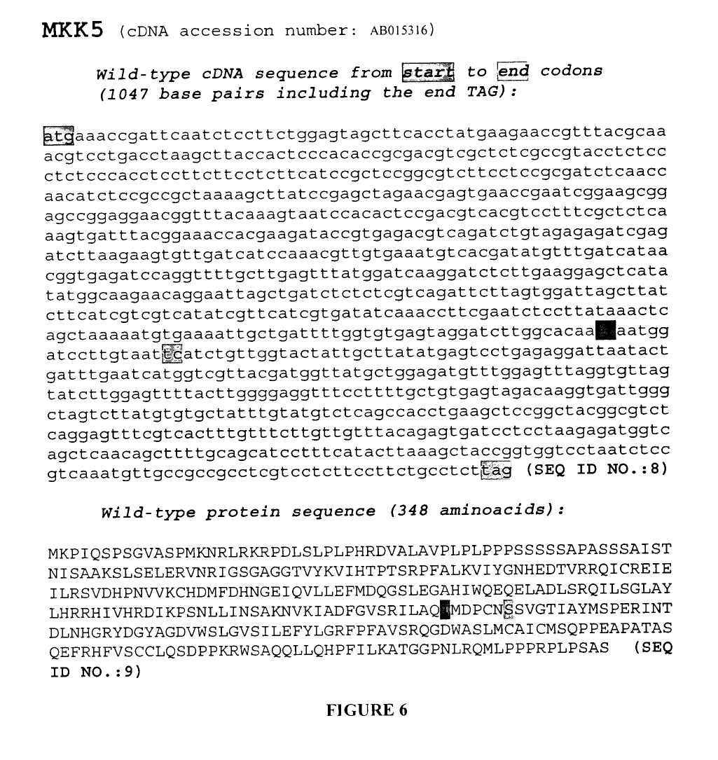

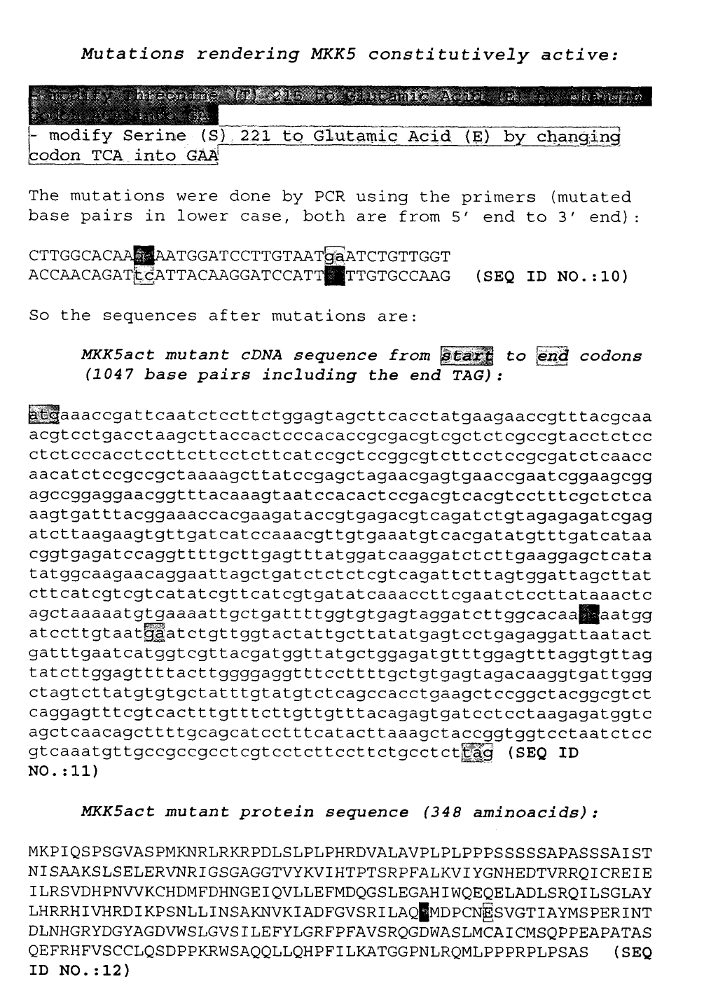

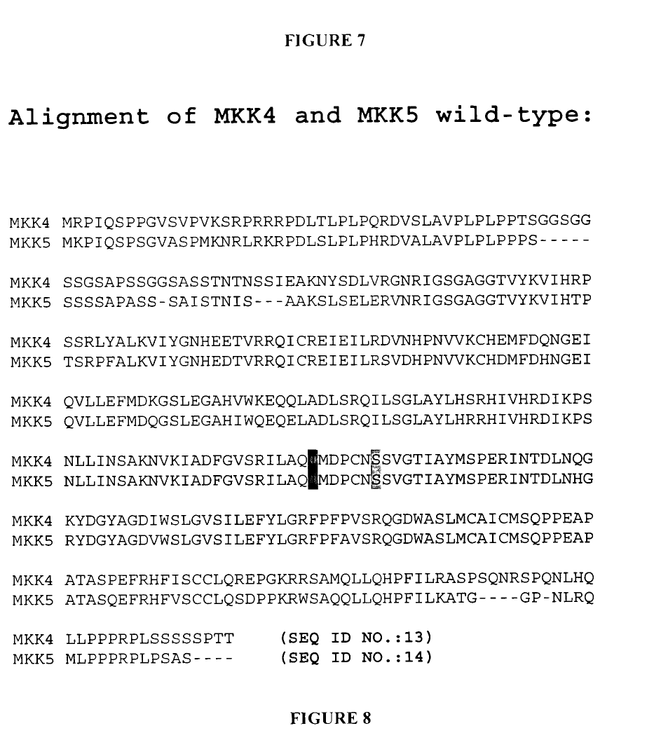

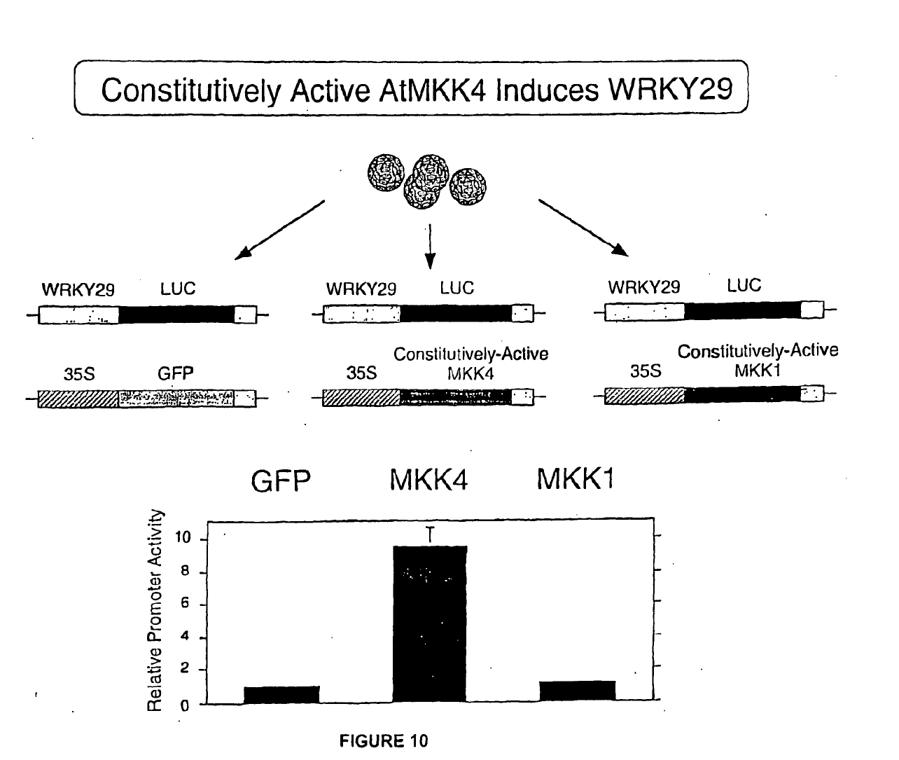

To determine whether MKK4 and MKK5 are involved in activating plant defense response genes, the effect of a constitutively active AtMKK4 and MKK5 on the activity of the WRKY29 promoter was examined. The nucleotide and predicted amino acid sequences of MKK4 and MKK5 are shown in FIGS. 4 and 6, respectively. The strategy for rendering each of these PKs constitutively active is also shown FIGS. 5 and 7. Furthermore, an alignment of MKK4 and MKK5 is shown in FIG. 8. The expression of constitutively active MKK4 and MKK5 was found to activate PAL1 and WRKY29 gene expression (FIG. 10).

### EXAMPLE 6

### WRKY29 Induces its Own Promoter

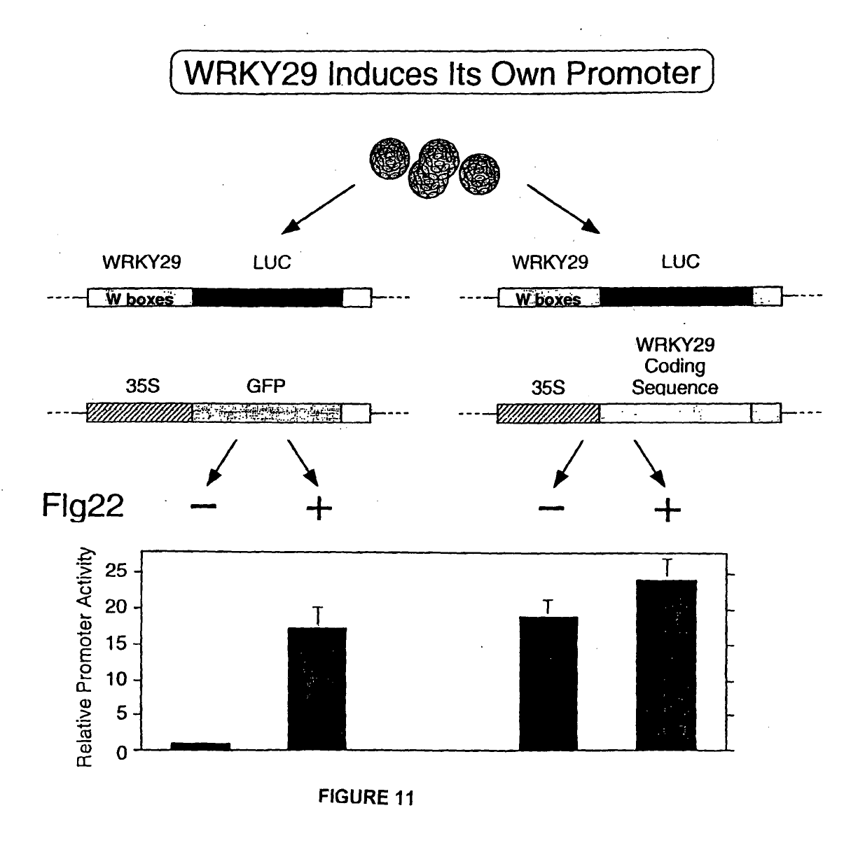

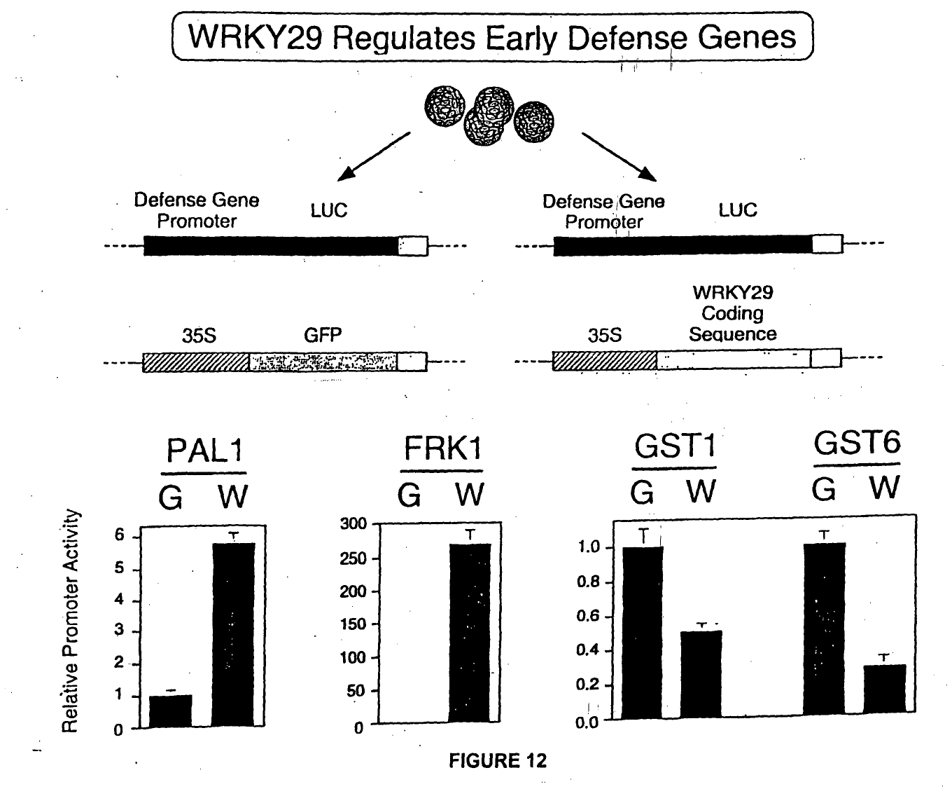

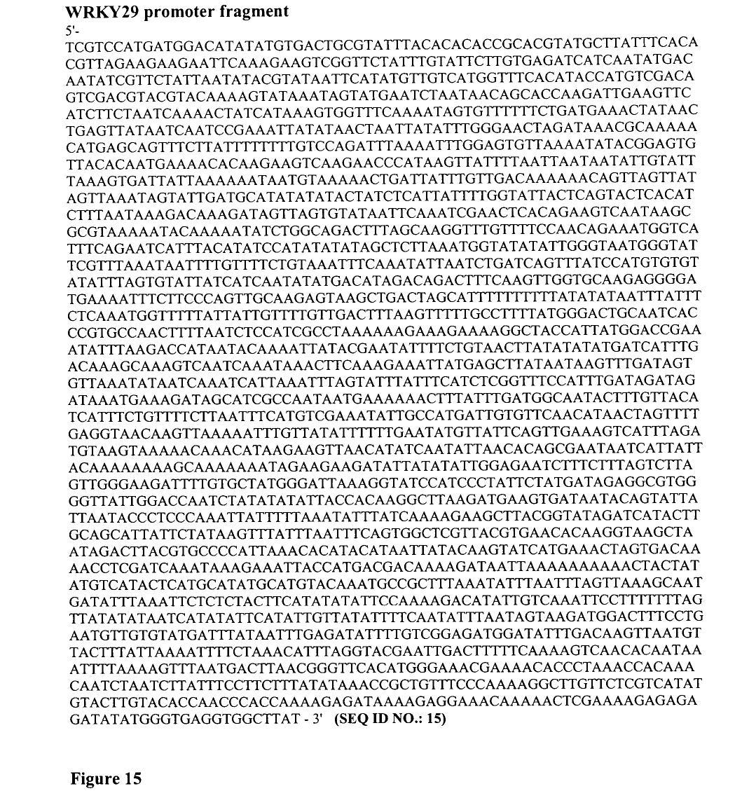

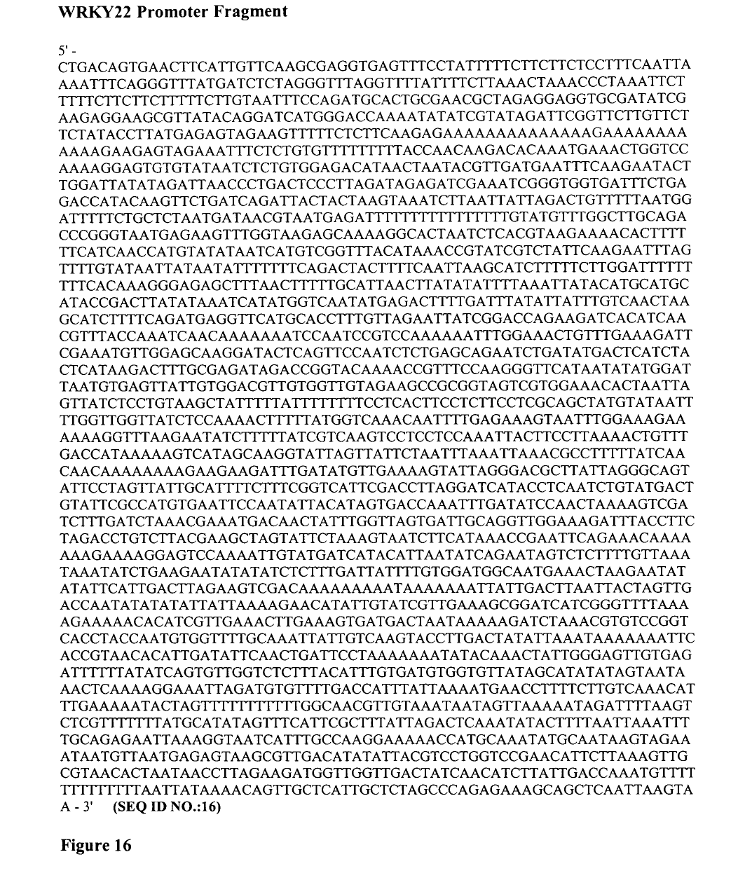

To determine the role of WRKY29 in plant defense, the effect of expressing WRKY29 on the activation of a reporter gene under the transcriptional control of the WRKY29 promoter was examined. As shown in FIG. 11, the WRKY29 polypeptide was found to induce expression of its own gene promoter. Exemplary pathogen-inducible WRKY promoter fragments are shown in FIGS. 15 and 16. In addition, as shown in FIG. 12, WRKY29 was found to regulate PAL1 and the so-called flg22 induced receptor-like kinase, but not a GST gene promoter.

### EXAMPLE 7

### Transient Expression of WRKY29 Reduces Susceptibility to Pseudomonas syringae

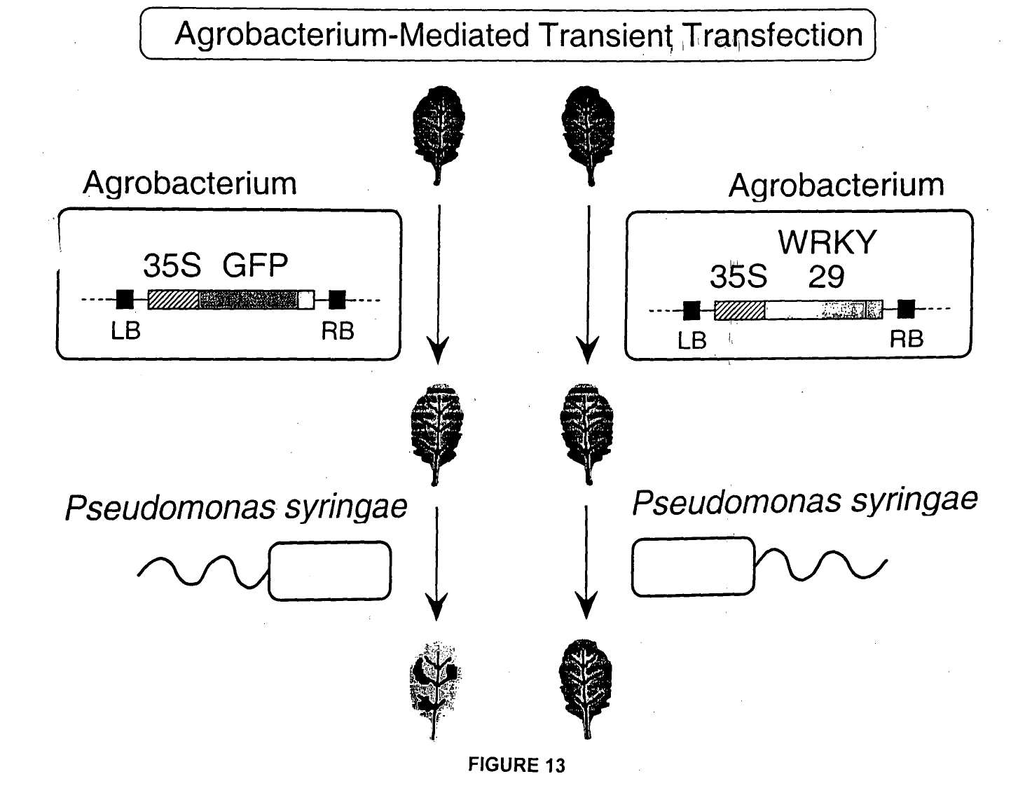

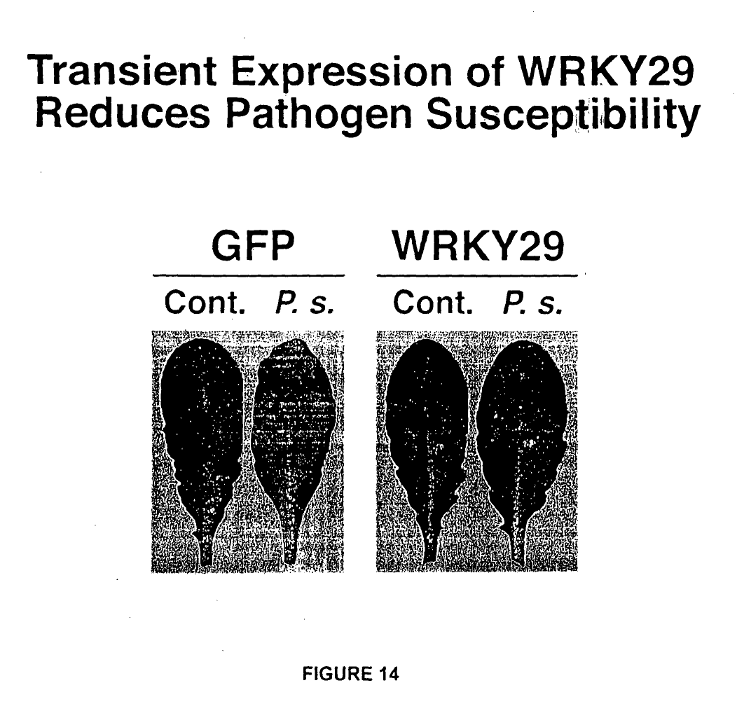

To determine whether expression of WRKY29 reduces pathogen susceptibility, Arabidopsis leaves from 4-5 week-old plants were infiltrated with Agrobacteria carrying 35S-GFP, 35S-MKK4act, or 35S-WRKY29 (FIG. 13). The plants were incubated for three days and the Agrobacterium-infiltrated leaves were further infiltrated with Pseudomonas syringae pv. maculicola strain ES4326 (resuspended in 10 mM MgSO₄, OD600=0.002) or 10 mM MgSO₄. The plants were incubated for two more days and the infiltrated leaves were cut for examination. As shown in FIG. 14, transient expression of WRKY29 was found to reduce susceptibility to Pseudomonas syringae pv. maculicola strain ES4326. Using the same procedure, it was also found that transient expression of a constitutively active MKK4 reduced the plant's susceptibility to Pseudomonas syringae.

### EXAMPLE 8

### Early Defense Gene Transcription Induced by flg22 in Arabidopsis Leaf Cells

To dissect the early signal transduction pathways in plant innate immune responses, we first established a leaf cell assay based on flg22 inducible transcription of early response genes in Arabidopsis mesophyll protoplasts. Few reporter genes have been developed for the dissection of the early stages of defense signaling pathways in plants (Durrant et al., Plant Cell 12:963-977, 2000; Maleck et al., Nature Genet. 26:403-410, 2000; Schenk et al., Proc. Natl. Acad. Sci., U.S.A. 97:11655-11660, 2000). To identify genes that are induced by flg22-activated defense signaling, we created a subtracted cDNA library that represented mRNA species induced at various times by the elicitor flg22 in Arabidopsis mesophyll protoplasts according to the methods described by Asai et al. (Plant Cell 12:1823-1835, 2000). Using flg22 rather than a pathogen or a natural elicitor avoided the possibility that multiple elicitors could be functioning in parallel. Furthermore, synchronous elicitation by flg22 is achieved more reproducibly in homogeneous mesophyll protoplasts than in intact leaves. In the library we found well-described defense genes, such as PAL1 (At2g37040), GST1 (At1g02930), PR1 (At2g19990), and PR5 (At1g75040), which are induced by a variety of pathogens and elicitors at different stages of the defense response in many plant species (Maleck et al., Nature Genet. 26:403-410, 2000; Schenk et al., Proc. Natl. Acad. Sci., U.S.A. 97:11655-11660, 2000; Asai et al., Plant Cell 12:1823-1835, 2000). These genes were also induced in Arabidopsis leaves infiltrated with flg22 (data not shown), suggesting that similar defense responses are induced by flg22 in isolated leaf protoplasts and in leaves of intact plants.

To develop new reporter genes that are expressed early in the primary defense response, we focused on flg22-activated genes identified in the subtraction library that were expressed at early time points (see below) and encode putative regulatory factors, including WRKY29 (At4g23550) (Euglem et al., Trends Plant Sci. 5:199-206, 2000) and FRK1 (FLG22-INDUCED RECEPTOR-LIKE KINASE 1, At2g19190), which encode a WRKY transcription factor (with a conserved WRKY DNA-binding domain) and a LRR receptor kinase, respectively. Induction of WRKY transcription factors and LRR receptor kinase genes by pathogens, pathogen-derived elicitors, or salicylic acid has been demonstrated in the leaves of several plant species including Arabidopsis (data not shown). Taken together, these results indicate that Arabidopsis protoplasts treated with flg22 increase transcription of a variety of defense-related genes that are also expressed in intact plants after pathogen infection.

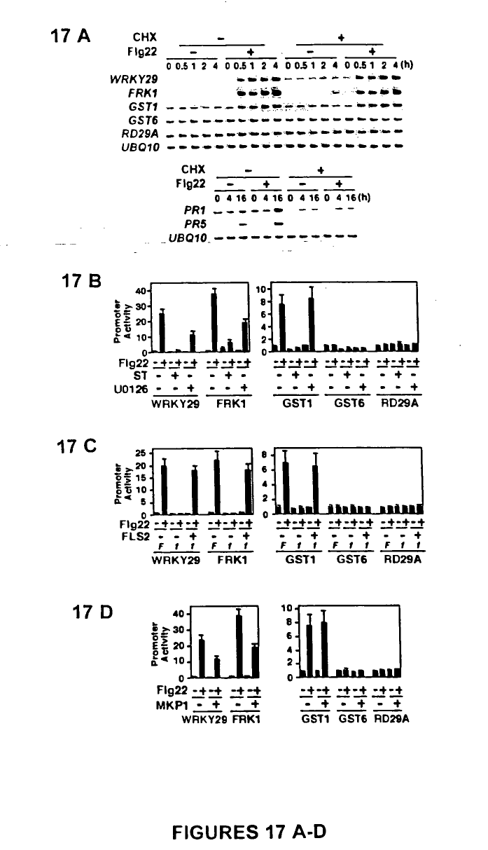

Polymerase chain reaction after reverse transcription of RNA (RT-PCR) analysis showed that WRKY29, FRK1, and GST1 mRNA levels were elevated in Arabidopsis protoplasts within 30 minutes after flg22 treatment (FIG. 17A), whereas induction of the extensively studied PR1 and PR5 genes occurred much later (FIG. 17A). Two stress-regulated genes, H₂O₂-inducible GST6 (At2g47730) and ABA-, cold-, or drought-responsive RD29A (AT5g52310) (Kovtun et al., Proc. Natl. Acad Sci., U.S.A. 97:2940-2945, 2000), were not induced by flg22 over basal levels of expression (FIG. 17A). In contrast to the common assumption that protoplasts isolated from fresh leaves are badly damaged, stressed and dying, or are already activated in defense responses, the data in FIG. 17 (as well as the data presented below) show that Arabidopsis mesophyll protoplasts respond specifically to flg22, similarly to the response observed in intact plants (Gomez-Gomez et al., Plant J. 18:277-284, 1999). The specific response of mesophyll protoplasts to flg22 is consistent with our previous studies using mesophyll protoplasts as a versatile system to analyze diverse stress signaling pathways (Kovtun et al., Proc. Natl. Acad Sci., U.S.A. 97:2940-2945, 2000; Sheen et al, Science 274:1900-1902, 1996) and cell death programs (Asai et al., Plant Cell 12:1823-1835, 2000).

To examine whether de novo protein synthesis is required for flg22-induced defense gene expression, protoplasts were preincubated with the protein synthesis inhibitor cycloheximide (CHX) and then treated with flg22. RT-PCR analysis showed that induction of the early response genes WRKY29, FRK1, and GST1 by flg22 was not significantly affected by CHX, whereas the inhibitor blocked induction of the late response genes PR1 and PR5, but not the expression of other control genes (FIG. 17A). To determine whether the early defense genes are activated transcriptionally, the promoters of the WRKY29, FRK1, GST1, GST6, and RD29A genes were fused to the LUC reporter gene as described by Kovtun et al. (Proc. Natl. Acad. Sci., USA 97:2940-2945, 2000) and tested for their response to flg22 in transiently transfected protoplasts. Consistent with the RT-PCR data obtained with the endogenous genes, the WRKY29, FRK1, and GST1 promoters, but not the GST6 and RD29A promoters, were induced by flg22 (FIG. 17B). In a parallel control experiment, the 23-amino acid peptide flg23RM, corresponding to the flg22-homologous sequence of the Rhizobium melliloti flagellin protein, activated none of these promoters (data not shown). R. melliloti is a plant symbiont that fails to activate (and/or suppress) host innate immunity. In previous work, R. melliloti flagellin was shown not to activate host defense responses (Felix et al., Plant J. 18:265-276, 1999; Gomez-Gomez et al., Plant J. 18:277-284, 1999). These results suggest that in response to pathogen-derived flagellin, WRKY29, FRK1, and GST1 are rapidly induced by pre-existing signaling molecules. Because the H₂O₂-responsive gene GST6 (Kovtun et al., Proc. Natl. Acad. Sci., U.S.A. 97:2940-2945, 2000) was not induced by flg22 (FIGS. 17A and 17B), it is likely that although flg22 induces an oxidative burst in Arabidopsis leaves (Gomez-Gomez et al., Mol. Cell 5:1003-1011, 2000; Gomez-Gomez et al., Plant J. 18:277-284, 1999), it may not elicit a strong or persistent oxidative burst in protoplasts. Consistent with this conclusion, an inhibitor of H₂O₂ accumulation, diphenylene iodonium, had no effect on flg22-induced WRKY29, FRK1, and GST1 gene expression (data not shown). Thus, the flg22-protoplast system distinguishes between flagellin and oxidative-stress signaling pathways (see below). In addition to the well-established parsley protoplast system derived from suspension cultures (Nurnberger et al., Trends Plant Sci. 6:372-379, 2001; Blume et al., Plant Cell 12:1425-1440, 2000; Hirt et al., Results and Problems in Cell Differentiation. MAP Kinases in Plant Signal Transduction (ed. Hirt, H.) 85-93 (Springer, Heidelberg, 2000); Ligterink et al., Science 276:2054-2057, 1997; Eulgem et al., EMBO J. 18:4689-4699, 1999), the Arabidopsis mesophyll protoplast transient expression system offers a new tool to study defense signaling based on early gene transcription.

### EXAMPLE 9

### Flg22 Signals Through FLS2 in Arabidopsis Protoplasts

To determine whether the WRKY29, FRK1, and GST1 promoters were activated by flg22 through the FLS2 LRR receptor kinase (Gomez-Gomez et al., Mol. Cell 5:1003-1011, 2000), protoplasts isolated from fls2 mutant leaves were transiently transfected with the WRKY29-LUC, FRK1-LUC, and GST1-LUC, reporter constructs. In the fls2 mutant protoplasts, none of the three promoters could be activated by flg22 (FIG. 17C). Transient expression of wild-type FLS2 (Gomez-Gomez et al., Mol. Cell 5:1003 -1011, 2000) restored flg22-inducibility of the promoters in the fls2 mutant protoplasts (FIG. 17C). Consistent with these results, the broad-spectrum protein kinase inhibitor staurosporine (ST) effectively blocked the ability of flg22 to activate the WRKY29, FRK1, and GST1 promoters (FIG. 17B). These data showed that flagellin signaling leading to the expression of WRKY29, FRK1, and GST1 requires FLS2.

### EXAMPLE 10

### Specific MAPKs in flg22 Signaling

The role of MAPK signaling in the flg22-mediated activation of the WRKY29, FRK1, and GST1 promoters was tested by using the MAPKK inhibitor U0126. As shown in FIG. 17B, U0126 partially blocked transcription of the WRKY29 and FRK1 promoters, but had no effect on the GST1 promoter. To further test the involvement of MAPK signaling in the activation of WRKY29 and FRK1 promoters, a mouse MAPK phosphatase (MKP1) was transiently expressed in Arabidopsis protoplasts (Kovtun et al., Nature 395:716-20, 1998). Similar to U0126, MKP1 partly reduced flg22-induced activation of WRKY29 and FRK1 promoters, but had no effect on the GST1 promoter (FIG. 17D). These results suggested that both MAPK-dependent and MAPK-independent signaling pathways act downstream of FLS2 to activate the WRKY29 and FRK1 promoters.

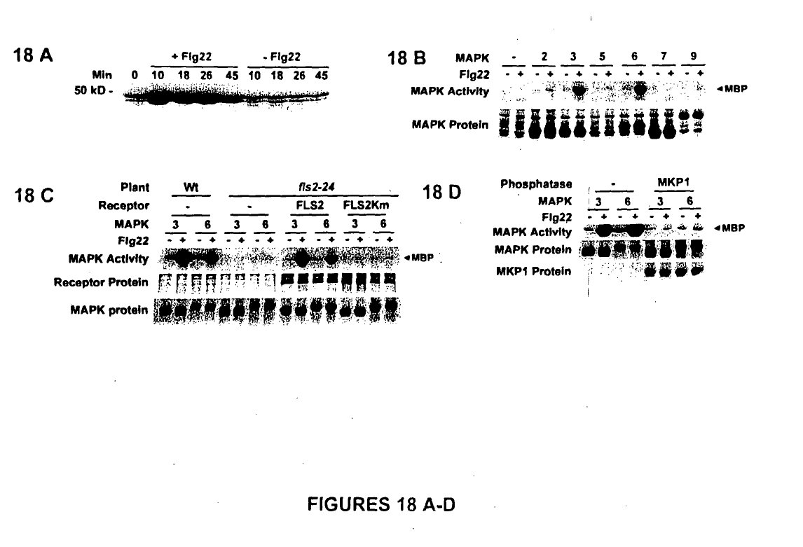

Since flg22 activation of WRKY29 and FRK1 promoters was partially blocked by U0126 and MKP1, we examined whether flg22 can activate MAPKs in Arabidopsis protoplasts. Treatment of protoplasts with flg22 resulted in rapid activation of endogenous protein kinases (PKs) that phosphorylated myelin basic protein (MBP), a commonly used MAPK substrate (FIG. 18A). These PKs were not activated by treatment with flg23RM (data not shown), suggesting their importance in pathogen defense. To systematically test the Arabidopsis MAPKs (MPKs) to determine which one(s) are involved in flg22 signaling, we chose six representative MPKs corresponding to four out of the five MPK subfamilies that may exhibit distinct functions based on sequence homology analysis (Tena et al., Curr. Opin. Plant Biol. 4:392-400, 2001; Mizoguchi et al, Results and Problems in Cell Differentiation. MAP Kinases in Plant Signal Transduction (ed. Hirt, H.) 29-38 (Springer, Heidelberg, 2000)). These MPKs were tagged with the haemagglutinin (HA) epitope, transiently expressed in protoplasts, immunoprecipitated with an anti-HA antibody, and tested in vitro for kinase activity (Kovtun et al., Proc. Natl. Acad. Sci., U.S.A. 97:2940-2945, 2000). The results indicated that MPK3 and MPK6, which belong to the same subfamily, but not the other MPKs, showed strong MBP phosphorylation following flg22 treatment (FIG. 18B). The involvement of MPK5 cannot be ruled out, however, due to its poor expression and/or instability in mesophyll protoplasts. The result is consistent with the previously reported activation of MPK6 in Arabidopsis cultured cells and leaf strips by flg22 (Nuhse et al., J. Biol. Chem. 275:7521-7526, 2000). The activation of MPK3 and MPK6 by flg22 did not occur in the fls2 mutant protoplasts unless functional FLS2 was co-expressed (FIG. 18C). Importantly, when a kinase inactive mutant of FLS2 (FLS2Km) was expressed in the fls2 mutant protoplasts, flg22-triggered activation of MPK3 and MPK6 was not observed (FIG. 18C), suggesting that the kinase activity of FLS2 is important for flagellin signaling and MAPK activation. Furthermore, the co-expression of MKP1, which partially blocked flg22-induced activation of WRKY29 and FRK1 promoters (FIG. 17D), eliminated MPK3 and MPK6 activation by flg22 (FIG. 18D). These results suggest that flagellin perception by FLS2 leads to the activation of MPK3 and/or MPK6. The studies also revealed the importance of MAPK-independent pathway in flg22 signaling.

### EXAMPLE 11

### Redundant MAPKKs in flg22 Signaling

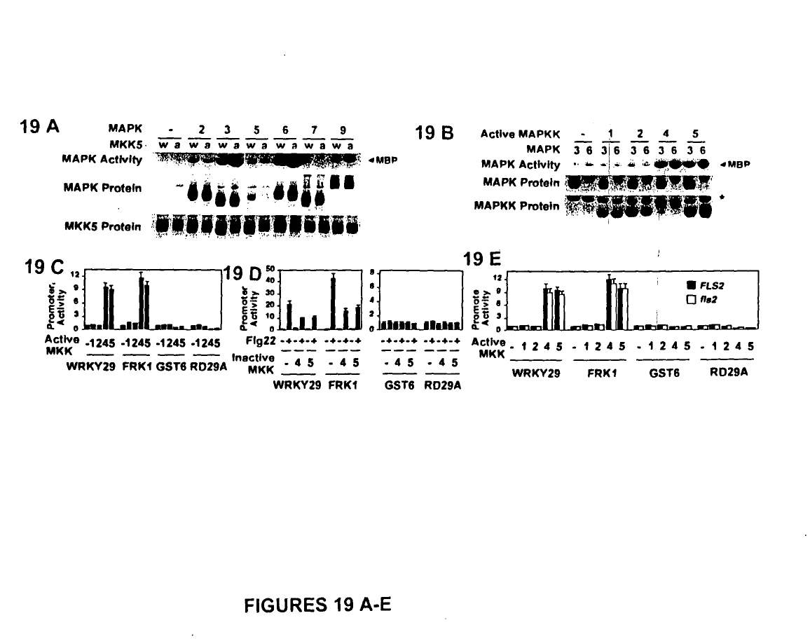

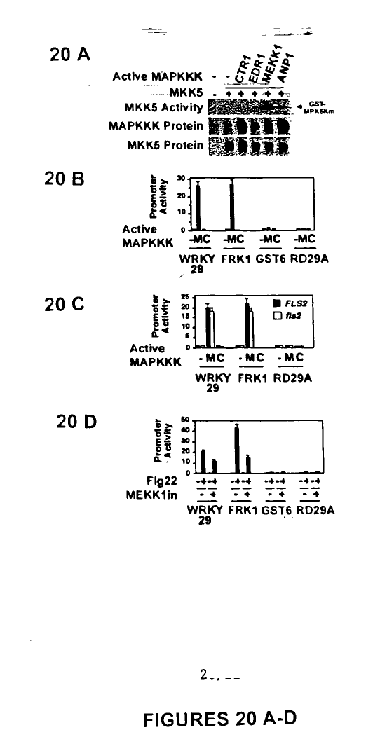

The Arabidopsis genome contains nine MAPKK (MKK) genes that belong to four subfamilies, suggesting at least four distinct functions (Tena et al., Curr. Opin. Plant Biol. 4:392-400, 2001). Four of these MKKs, MKK1, MKK2, MKK4, and MKK5, belonging to two subfamilies, are expressed in leaf cells (Tena et al., Curr. Opin. Plant Biol. 4:392-400, 2001; Mizoguchi et al, Results and Problems in Cell Differentiation: MAP Kinases in Plant Signal Transduction (ed. Hirt, H.) pp. 29-38 (Springer, Heidelberg, 2000)). Therefore, these four MKK genes were cloned and analyzed in the protoplast transient expression assay. To distinguish MKKs from the HA-tagged MPKs in transfected protoplasts, a MYC-epitope tag was fused to each MKK. Since MAPKKs require phosphorylation to be activated, gain-of-function mutants of the four MKKs were generated by converting the conserved serine and/or threonine residues in the kinase-activation loop located between subdomains VII and VIII to aspartate or glutamate to mimic phosphorylation. The MYC-tagged wild-type or constitutively active MKKs were co-expressed individually with the six HA-tagged MPKs to systematically determine their regulatory relationships. The results showed that constitutively active MKK4 and MKK5 (MKK4a and MKK5a, respectively), which belong to the same subfamily, were equally effective at phosphorylating (FIG. 20A) and activating MPK3 and MPK6 (FIGS. 19A and 19B). We cannot rule out the possibility that MPK5 is also activated by MKK4a and/or MKK5a since the expression of MPK5 was poor (FIG. 19A). In contrast, constitutively active MKK1 and MKK2 (MKK1a and MKK2a, respectively) were unable to activate these MPKs (FIG. 19B), although MKK1a did activate other MPKs (data not shown).

To directly test the role of MKK4 and MKK5 in flg22 signaling, plasmid DNA expressing the constitutively active MKK constructs was cotransfected with various reporter gene constructs into protoplasts (Kovtun et al., Proc. Natl. Acad. Sci., U.S.A. 97:2940-2945, 2000). Either MKK4a or MKK5a, but not MKK1a or MKK2a, was sufficient to specifically activate the WRKY29 and FRK1 promoters (FIG. 19C). To further test the importance of MKK4 and MKK5 in the early defense response, corresponding dominant-negative mutants were generated by replacing the conserved lysine residue in the ATP binding site with methionine. As shown in FIG. 19D, dominant-negative MKK4 or MKK5 but not MKK1 (data not shown) could partially block flg22 activation of the WRKY29 and FRK1 promoters similarly to U0126 or MKP1 (FIGS. 17B and 17D). In addition, MKK4a and MKK5a, but not MKK1 a and MKK2a, could bypass the requirement of FLS2 in flg22 signaling, demonstrated by their ability to activate the WRKY29 and FRK1 promoters in fls2 mutant protoplasts (FIG. 19E).

Thus, on the basis of both gain-of-function and loss-of-function analyses, it appears that flagellin signaling activates MKK4 and MKK5, which in turn phosphorylate and activate MPK3 and MPK6, leading to the expression of early defense response genes. The functions of MKK4 and MKK5 in flg22 signaling are likely redundant in Arabidopsis leaf cells (see below). It has been proposed that MPK3 and/or MPK6 mediate expression of GST6 in Arabidopsis protoplasts treated with H₂ O₂ (Kovtun et al., Proc. Natl. Acad. Sci., U.S.A. 97:2940-2945, 2000). A MAPKK functioning in this oxidative-stress signaling pathway has not been identified. Although MKK4 and MKK5 can activate MPK3 and MPK6, neither MKK4a nor MKK5a was able to induce the expression of GST6, a reporter gene in the oxidative stress signaling pathway (FIGS. 19C and 19E). This result suggests that flagellin and H₂O₂ may activate different MAPK signaling cascades.

### EXAMPLE 12

### A Specific MAPKKK in flg22 Signaling

On the basis of sequence homology in the kinase domains to mammalian MAPKKKs, there are at least 25 putative Arabidopsis MAPKKKs that can be divided into six subgroups (Tena et al., Curr. Opin. Plant Biol. 4:392-400, 2001). The sequence similarity among the subgroups is low, especially in the regulatory domains, indicating that the subgroups have distinct functions (Tena et al., Curr. Opin. Plant Biol. 4:392-400, 2001; Jouannic et al., Gene 233:1-11, 1999). We analyzed four Arabidopsis MAPKKKs, CTR1, EDR1, MEKK1, and ANP1, from three subgroups expressed in leaf cells, to determine whether any of them could activate MKK5. Constitutively active derivatives of these four MAPKKKs were constructed by deleting the putative regulatory domain (Kovtun et al., Proc. Natl Acad. Sci., U.S.A. 97:2940-2945, 2000). The four constitutively active MAPKKKs were fused to a HA-epitope tag and coexpressed individually with MYC-epitope-tagged wild-type MKK5 in protoplasts. To determine whether MKK5 had been phosphorylated and activated, MKK5 was immunoprecipitated using an anti-MYC antibody, and its activity was determined in vitro using a purified kinase inactive MPK6 fused to glutathione S-transferase (GST-MPK6Km) as a substrate. As shown in FIG. 20A, MKK5 was specifically activated by constitutively active MEKKb1 (ΔMEKK1) and could phosphorylate MPK6. Constitutively active ANP1 (ΔANP1), which can mimic the H₂O₂ signal and induce the GST6 promoter (Kovtun et al., Proc. Natl. Acad. Sci., U.S.A. 97:2940-2945, 2000), activated MKK5 only marginally despite a high level of protein expression (FIG. 20A), supporting the idea that flagellin and H₂O₂ activate different MAPK pathways.

Consistent with the above results, ΔMEKK1 activated the WRKY29 and FRK1 promoters in the absence of flg22 in both wild-type and fls2 mutant protoplasts (FIGS. 20B and 20C), but not the activity of the H₂O₂- or ΔANP1 -inducible GST6 promoter (FIG. 20B). Furthermore, constitutively active CTR1 (ΔCTR1), which did not activate MKK5, had no effect on the WRKY29 and FRK1 promoter activities (FIGS. 20B and 20C). To further test the involvement of MEKK1 in flg22 signaling, a dominant-negative mutant was generated by altering the ATP binding site, but maintaining the regulatory and protein-protein interaction domains of the full-length MEKK1. The dominant-negative mutant (MEKK1in) partially suppressed flg22 activation of WRKY29 and FRK1 promoters (FIG. 20D), similarly to U0126 treatment, MKP1, and dominant-negative MKK4 or MKK5 (FIGS. 17B, 17D, and 19D). Dominant negative CTR1 did not have an effect (data not shown). Taken together, the results described so far strongly suggest that flagellin perception by FLS2 leads to the induction of WRKY29 and FRK1 transcription through the activation of a MAPK signaling cascade consisting of MEKK1, MKK4/MKK5, and MPK3/MPK6.

### EXAMPLE 13

### Positive Feedback Control by WRKY Transcription Factors

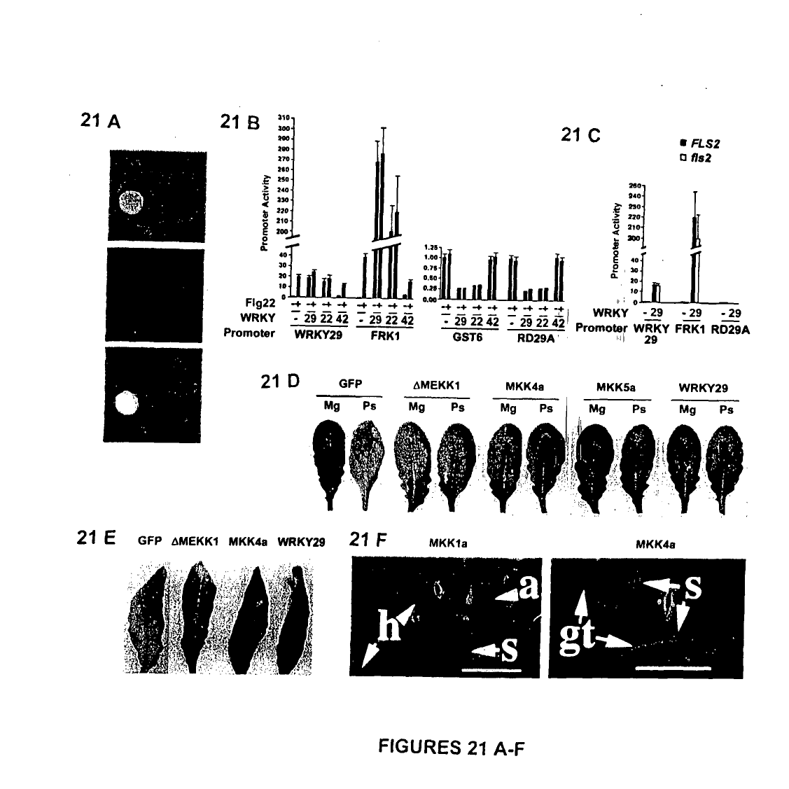

WRKY proteins have been shown to bind to W box DNA elements (containing a TGAC core sequence) that are found in the promoters of many defense-related genes including WRKY29 (10 W boxes) and FRK1 (15 W boxes) (Maleck et al., Nature Genet. 26:403-410, 2000; Euglem et al., Trends Plant Sci. 5:199-206, 2000; Du et al., Plant J. 24:837-847, 2000). In parsley protoplasts, a fungal pathogen-inducible WRKY1 protein, with two WRKY domains, is targeted to the nucleus and activates its own promoter by binding to multiple W boxes (Eulgem et al., EMBO J. 18:4689-4699, 1999). Arabidopsis WRKY29, with a single WRKY domain, is not the ortholog of parsley WRKY1 (Eulgem et al., EMBO J. 18:4689-4699, 1999), and is the WRKY gene shown to be induced by pathogen-derived flagellin (FIG. 17). WRKY29-GFP, a WRKY29 and green fluorescent protein fusion, was constitutively localized in the nucleus in the presence or absence of flg22 (FIG. 21A). Transient expression of WRKY29 activated its own promoter in the absence of flg22 (FIG. 21B), indicating a positive feedback control (Eulgem et al., EMBO J. 18:4689-4699, 1999). WRKY29 also strongly activated the FRK1 promoter, but suppressed the basal activities of the GST6 and RD29A promoters (FIG. 21C). Transient expression of WRKY29 activated the WRKY29 and FRK1, but not RD29A, promoters in the fls2 mutant protoplasts, suggesting that WRKY29 acts downstream of the flagellin receptor (FIG. 21C). Treatment of WRKY29-expressing protoplasts with flg22 did not further enhance the activity of the WRKY29 and FRK1 promoters (FIG. 21B). These results are consistent with the idea that unmodified wild-type WRKY29 can act as a transcriptional activator. In contrast to WRKY29, a different Arabidopsis WRKY protein, WRKY42 (At4g04450), did not activate the WRKY29 and FRK1 promoters, but rather slightly inhibited the flg22 activation of these promoters (FIG. 21B). Moreover, unlike WRKY29, WRKY42 did not suppress basal activities of the GST6 and RD29A promoters (FIG. 21B). Thus, we postulated that WRKY29 is a key transcriptional activator involved in the expression of defense genes in Arabidopsis innate immune responses. Similarly to MKK and MPK gene families, the large WRKY family also has members that are highly homologous to WRKY29. The homologous WRKY22 (At4g01250), in the same WRKY subgroup (Euglem et al., Trends Plant Sci. 5:199-206, 2000), was cloned and tested in the protoplast transient assay. WRKY22 regulated these promoters similarly to WRKY29 (FIG. 21B), suggesting that WRKY29 and WRKY22 may be functionally redundant.

### EXAMPLE 14

### Resistance to Bacterial and Fungal Pathogens

On the basis of the results obtained with the protoplast transient expression assays, we tested whether Arabidopsis plants exhibited enhanced resistance to bacterial pathogens when the flagellin MAPK cascade is constitutively activated or WRKY29 is expressed by means of Agrobacterium-mediated transient transformation (Xiang et al., Plant Mol. Biol. 40:711-717, 1999). This method has allowed a reliable assessment of the effect of transgenes in leaves (Nimchuk et al., Cell 101 :353-363, 2000; Swiderski et al., Plant J 26:101-12, 2001; Yang et al., Proc. Natl. Acad. Sci., U.S.A. 98:741-746, 2001). When infected by the virulent bacterial pathogen Pseudomonas syringae, transiently transformed control leaves expressing GFP developed chlorotic lesions (FIG. 21D). Similar results were obtained with untransformed leaves and leaves expressing ΔCTR1, MKK1a, or wild-type WRKY42 as controls (not shown). In contrast, transformed leaves expressing ΔMEKK1, MKK4a, MKK5a, or wild-type WRKY29 displayed enhanced resistance to P. syringae (FIG. 21D). The observation that both MKK4a and MKK5a can confer pathogen resistance is consistent with the idea that these MAPKKs are functionally redundant.

Importantly, development of soft-rot symptoms caused by infection with the fungal pathogen Botrytis cinerea was also effectively suppressed when ΔMEKK1, MKK4a, or WRKY29, but not GFP, was transiently expressed in leaves (FIG. 21E). Microscopic analysis indicated that in Arabidopsis leaves expressing MKK1a as a control, fungal spores germinated and formed superficial mycelium and branched appressoria filled with intensely stained cytoplasm within two days after B. cinerea infection (FIG. 21F). In contrast, only germ tubes were formed during the same period of time in leaves expressing MKK4a. Similar results were obtained with ΔMEKK1, MKK5a, and WRKY29 (data not shown). These results suggest that defense responses activated by the flagellin MAPK cascade or WRKY29 are specifically effective against fungal as well as bacterial pathogens.

In addition, transient expression of MKK4a in Arabidopsis leaves, as in FIG. 21D, inhibited the growth of P. syringae maculicola ES4326 15-fold one day after infiltration, and over 100-fold four days after infiltration. The results are consistent with the leaf phenotype shown in FIG. 21D.

Examples 8-14 described above were performed using the following methods.

Reporter Constructs

To identify early response genes, a subtracted cDNA library that represented mRNA species induced by the elicitor flg22 in Arabidopsis mesophyll protoplasts was generated as previously described (Asai et al., Plant Cell 12:1823-1835, 2000). The DNA regions immediately upstream from the translation start sites of the WRKY29, FRK1, and GST1 genes were amplified by PCR from Arabidopsis (Col-0) genomic DNA. The sizes of the amplified fragments were 2.6 kb, 2.8 kb, and 0.9 kb, respectively. These promoters were fused to a luciferase (LUC) reporter gene to generate WRKY29-LUC, FRK1-LUC, and GST1-LUC as described by Kovtun et al., (Proc. Natl. Acad. Sci., U.S.A. 97:2940-2945, 2000).

Effector Constructs

Arabidopsis cDNAs, encoding MAPKs: MPK2 (At1g59580), MPK3 (At3g45640), MPK5 (At4g11330), MPK6 (At2g43790), MPK7 (At2g18170), MPK9 (At3g18040); MAPKKs: MEK1 (At4g26070), MKK2 (At4g29810), MKK4 (At1g51660), MKK5 (At3g21220); MAPKKKs: CTR1 (At5g03730), EDR1(At1g08720), MEKK1 (At4g08500), ANP1 (At1g090000); receptor FLS2 (At5g46330); transcription factors WRKY22 (At4g01250), WRKY29 (At4g23550), and WRKY42 (At4g04450), were obtained by PCR from an Arabidopsis cDNA library and verified by sequencing. Mouse MKP1 phosphatase was a gift from H. Sun and N. Tonks (Kovtun et al., Nature 395:716-20, 1998). PCR products were fused to the double HA (MAPK, MAPKKK, receptor and WRKY) or MYC (MAPKK and MKP1) epitope tag sequence and inserted into a plant expression vector or pCB302 containing the 35SC4PPDK promoter and the NOS terminator (Kovtun et al., Proc. Natl. Acad. Sci., U.S.A. 97:2940-2945, 2000; Kovtun et al., Nature 395:716-20, 1998; and Xiang et al., Plant Mol. Biol. 40:711-717, 1999).

The constitutively active forms of MAPKKs were generated by site-specific mutation, replacing the serine or threonine residues in the activation loop domain [S/T]XXXXX[S/T] by acidic amino acids glutamate or aspartate: MKK1(T218ES224D), MKK2(T220DT226E), MKK4(T224DS230E), and MKK5(T215ES221E). The active forms of MAPKKK were generated by keeping the catalytic domain only: ΔCTR1 (544-799), ΔEDR1(662-921), ΔMEKK1(326-592), ΔANP1(57-338). The inactive forms of all kinases were generated by site-specific mutation, replacing the conserved lysine residues in the kinase ATP binding loop by a methionine: FLS2(K898M), MKK4(K108M), MKK5(K99M), MPK6(K92M), MEKK1(K361M). All these mutations were verified by sequencing.

GST Fusion Proteins

The GST fusions were generated by subcloning the coding sequences of MAPKs and MAPKKs into pGEX-4T-1 vector (Amersham Pharmacia Biotech) in frame with the GST coding sequence. All the GST fusion proteins were purified based on the manufacture's procedure and subjected to in vitro phosphorylation assay. Except the kinase inactive mutants that were used as controls, all PKs showed significant autophosphorylation activity and were able to phosphorylate an exogenous substrate, indicating that they are active kinases (data not shown).

Arabidopsis mesophyll Protoplast Transient Expression Assay

The protoplast transient expression assay was carried out as described previously (Kovtun et al., Proc. Natl. Acad. Sci., U.S.A. 97:2940-2945, 2000). Promoter activities are represented by LUC/GUS activities, and normalized to the value obtained with protoplasts without the treatments. In the case of protoplasts prepared from fls2 mutant plants, promoter activities are normalized to the value obtained with wild-type protoplasts transfected with a control GFP plasmid. Constitutively active MAPKKKs, immunoprecipitated from transfected protoplasts, phosphorylated casein in vitro (Kovtun et al., Nature 395:716-20, 1998), indicating that they are active kinases. GFP fluorescence was observed by either Nikon TE200 fluorescent microscopy or with Leica TCSNT Confocal Spectrophotometer (Germany). Coexpression of a nuclear-targeted red-fluorescent protein was used as a control (Patharkar et al., Plant J. 24:679-691, 2000).

RT-PCR

Samples were taken at indicated time points after treatment and total RNA was prepared as described (Asai et al., Plant Cell 12:1823-1835, 2000). RT-PCR was performed with 1 ng of total RNA and 0.6 μM of each primer using OneStep RT-PCR Kit (Qiagen, Germany). PCR was run for 35 cycles. Constitutively expressed UBQ10 (AT4g05320) or ACTIN-1 (At2g37620) mRNA was always co-amplified with each mRNA and used as an internal standard. For these genes, 0.1 μM of each primer was used. Typical results for UBQ10 mRNA are shown.

Protein Kinase Assays

The MAPK in-gel kinase assay was carried out as described by Kovtun et al. (Proc. Natl. Acad Sci., U.S.A. 97:2940-2945, 2000). For immuno-complex assays, tagged kinases were immunoprecipitated from lysates of transfected protoplasts with the corresponding antibody and analyzed with a known substrate as indicated (MBP or GST-MPK6 Km).

Agrobacterium-Mediated Transient Transformation

To analyze plant susceptibility to a bacterial pathogen, five-week-old Arabidopsis plants were infiltrated with a suspension (containing 100 μM acetosyringone) of A. tumefaciens carrying a binary vector plasmid pCB302 (Xiang et al., Plant Mol. Biol. 40:711-717, 1999) expressing GFP, ΔMEKK1, MKK4a, MKK5a, or WRKY29. The plants were incubated for three days under the conditions as described (Asai et al., Plant Cell 12:1823-1835, 2000). Infiltrated leaves were then inoculated with 10 mM MgSO₄ or the same solution with suspended P. syringae maculicola ES4326 (10⁴ cfu/cm²) at the location of transient gene expression. The leaves were photographed three days later.

To analyze plant susceptibility to a fungal pathogen, a suspension (containing 100 μM acetosyringone and 0.01% Silwett L-77) of A. tumefaciens carrying a binary vector plasmid expressing GFP, ΔMEKK1, MKK4a, MKK1a, or WRKY29 was first applied to the lower surface of a leaf of six-week-old Arabidopsis. The leaves were incubated for 24 hours and then the upper surface of transformed leaves was infected with B. cinerea sclerotia. The infected leaves were photographed five days later after removing the fungal sclerotia. To observe the early stage of B. cinerea development, 5 μl drops of a 5×10⁵ spores/ml suspension were placed on the upper surface of an Arabidopsis leaf. Two days after infection, fungal structures were visualized by Trypan blue-lactophenol 25 staining and observed with Confocal Spectrophotometer TCSNT (Leica, Germany).

Untransformed and GFP-expressing control leaves developed similar lesions as the MKK1a-expressing leaves.

### SUMMARY

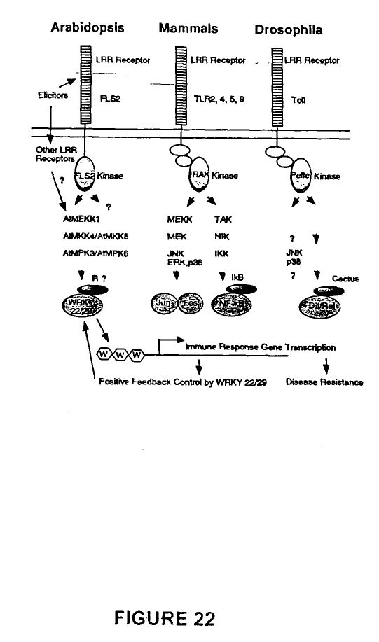

We have demonstrated that Arabidopsis mesophyll protoplast transient expression assays can be combined with genetic and genomic information to provide a powerful tool for the analysis of MAPK signaling involved in plant innate immunity. Importantly, the key components of the innate immune signaling pathway identified using the leaf cell system confer pathogen resistance when expressed in leaves of intact plants (FIG. 21). Since wild-type WRKY22 or WRKY29 is sufficient to mimic flg22 and MAPK signaling, we postulate that a specific WRKY inhibitor may be phosphorylated and inactivated by the flagellin MAPK cascade upon pathogen infection. As summarized in the model shown in FIG. 22, this would make the flg22 signaling pathway partially analogous to signaling pathways in insects and mammals, in which TLRs initiate a cascade of signaling events leading to phosphorylation and degradation of an inhibitor of a Rel-like transcription factor (Aderem et al., Nature 406:782-787, 2000; Khush et al., Trends Genet. 16:442-449, 2000). It will be interesting to test whether the flg22- and FLS2-dependent phosphorylation of the ankyrin-containing protein AtPhos43, recently identified by a proteomic approach (Peck et al, Plant Cell 13:1467-1475, 2001), is MAPK dependent and can control WRKY22 and WRKY29 sub-cellular localization and/or activity.

We further propose that early signaling events initiated by diverse pathogens converge into a conserved MAPK cascade that positively regulates plant defense responses (FIG. 22). This is consistent with the observation that similar plant defense responses are elicited by many viral, bacterial, and fungal pathogens. Because MAPK signaling components are highly conserved among diverse plant species, our results with flagellin elicitation provides insights into the genetic manipulation of resistance to a wide array of pathogens in economically important crop plants.

As shown in FIG. 17B, 17D, 19D, and 20D, our data also reveal the previously unexpected existence of a MAPK-independent pathway in flg22 signaling.

The lack of MAPK cascade mutations in the flg22 signaling pathway is most likely due to functional redundancy in the pathway, as suggested by the identification of the MKK4/MKK5 and MPK3/MPK6 pairs in our study. The WRKY22 and WRKY29 transcription factors in the flg22 signaling pathway may also provide redundant functions. Furthermore, similar to the yeast MAPKKK STE11 (Madhani et al., Trends Genet. 14:151-155, 1998), some plant MAPK signaling components could be involved in more than one pathway (Tena et al., Curr. Opin. Plant Biol. 4:392-400, 2001). The H₂O₂-activated MAPK cascade shares MPK3 and MPK6 with the flg22 pathway but activates different target genes (Kovtun et al., Proc. Natl. Acad. Sci., U.S.A. 97:2940-2945, 2000). The MAPKKK of the flagellin cascade, MEKK1, may also initiate a different MAPK cascade. In addition, it should be noted that, besides the flagellin MAPK cascade, other pathways utilizing untested MAPK, MAPKK, and MAPKKK genes could also play important roles in Arabidopsis innate immunity. Finally, plants transiently expressing ΔMEKK1 , MKK4a, or WRKY29 are resistant to both bacterial and fungal pathogens, suggesting that other pathogen-derived signals in addition to flg22 may activate the MEKK1, MKK4/MKK5, MPK3/MPK6, WRKY22/29 signaling pathway through distinct receptors (FIG. 22) (Peck et al, Plant Cell 13:1467-1475, 2001). Given this complexity, a combination of genetic, genomic, cellular, and biochemical approaches including the construction of knock-out mutants and expression of signaling components in transgenic plants will be required to untangle the intertwined signaling webs mediated by MAPK cascades in plants.

Methods for isolating sequences encoding MAPKKKs, MAPKKs, and WRKYs now follow. In addition, methods for expressing such sequences in a plant and methods for engineering disease resistant plants are also described.

Isolation of Sequences Encoding MAPKKKs, MAPKKs, and WRKYs

The isolation of additional MAPKKK (MAPKKs and WRKYs) coding sequences, as well as MAPKKK and MAPKK kinase domains, having the ability to regulate pathogen defense in plants is accomplished using standard strategies and techniques that are well known in the art.

In one particular example, the tobacco NPK1 sequences (or, for example, Arabidopsis ANP1, ANP2, or ANP3 sequences) may be used, together with conventional screening methods of nucleic acid hybridization screening, to isolate additional sequences encoding MAPKKK polypeptides (or kinase domain-containing fragments thereof), as well as kinase domains of MAPKKK. Such hybridization techniques and screening procedures are well known to those skilled in the art and are described, for example, in Benton and Davis, Science 196: 180, 1977; Grunstein and Hogness, Proc. Natl. Acad. Sci., USA 72: 3961, 1975; Ausubel et al. Current Protocols in Molecular Biology, Wiley Interscience, New York; Berger and Kimmel, Guide to Molecular Cloning Techniques, 1987, Academic Press, New York.; and Sambrook et al., Molecular Cloning. A Laboratory Manual, Cold Spring Harbor Laboratory Press, New York. In one particular example, all or part of the NPK1 gene may be used as a probe to screen a recombinant plant DNA library for genes having sequence identity or similarity to the NPK1 gene or its kinase domain. Hybridizing sequences are detected by plaque or colony hybridization according to the methods described below.

Alternatively, using all or a portion of the amino acid sequence of the kinase domain, one may readily design kinase domain-specific oligonucleotide probes, including kinase domain degenerate oligonucleotide probes (i.e., a mixture of all possible coding sequences for a given amino acid sequence). These oligonucleotides may be based upon the sequence of either DNA strand and any appropriate portion of the kinase domain sequence. General methods for designing and preparing such probes are provided, for example, in Ausubel et al., Current Protocols in Molecular Biology, Wiley Interscience, New York; and Berger and Kimmel, Guide to Molecular Cloning Techniques, 1987, Academic Press, New York. These oligonucleotides are useful for kinase domain sequence isolation, either through their use as probes capable of hybridizing to kinase complementary sequences or as primers for various amplification techniques, for example, polymerase chain reaction (PCR) cloning strategies. If desired, a combination of different oligonucleotide probes may be used for the screening of a recombinant DNA library. The oligonucleotides may be detectably-labeled using methods known in the art and used to probe filter replicas from a recombinant DNA library. Recombinant DNA libraries are prepared according to methods well known in the art, for example, as described in Ausubel et al. (supra), or they may be obtained from commercial sources.

As discussed above, kinase domain-specific oligonucleotides may also be used as primers in amplification cloning strategies, for example, using PCR. PCR methods are well known in the art and are described, for example, in PCR Technology, Erlich, ed., Stockton Press, London, 1989; PCR Protocols: A Guide to Methods and Applications, Innis et al., eds., Academic Press, Inc., New York, 1990; and Ausubel et al. (supra). Primers are optionally designed to allow cloning of the amplified product into a suitable vector, for example, by including appropriate restriction sites at the 5′ and 3′ ends of the amplified fragment (as described herein). If desired, kinase domain sequences may be isolated using the PCR “RACE” technique, or Rapid Amplification of cDNA Ends (see, e.g., Innis et al. (supra)). By this method, oligonucleotide primers based on an kinase domain sequence are oriented in the 3′ and 5′ directions and are used to generate overlapping PCR fragments. These overlapping 3′- and 5′-end RACE products are combined to produce an intact full-length cDNA. This method is described in Innis et al. (supra); and Frohman et al., Proc. Natl. Acad. Sci., USA 85: 8998, 1988.

Confirmation of a sequence's relatedness to the kinase domains of the NPK and ANP MAPKKKs may be accomplished by a variety of conventional methods including, but not limited to, sequence comparison of the gene and its expressed product. In addition, the activity of the gene product may be evaluated according to any of the techniques described.

Once a MAPKKK gene or its kinase domain (or a gene encoding a MAPKK or WRKY polypeptide) is identified, it is cloned according to standard methods and used for the construction of plant expression vectors as described below.

Expression Constructs

Those skilled in the field of molecular biology will understand that any of a wide variety of expression systems may be used to provide the recombinant protein. The precise host cell used is not critical to the invention. A MAPKKK polypeptide or its kinase domain (or a MAPKK or WRKY polypeptide) may be produced in a prokaryotic host, for example, E. coli, or in a eukaryotic host, for example, Saccharomyces cerevisiae, mammalian cells (for example, COS 1 or NIH 3T3 cells), or any of a number of plant hosts including, without limitation, algae, tree species, ornamental species, temperate fruit species, tropical fruit species, vegetable species, legume species, crucifer species, monocots, dicots, or in any plant of commercial or agricultural significance.

Particular examples of suitable plant hosts include, but are not limited to, Conifers, Petunia, Tomato, Potato, Tobacco, Arabidopsis, Lettuce, Sunflower, Oilseed rape, Flax, Cotton, Sugarbeet, Celery, Soybean, Alfalfa, Medicago, Lotus, Vigna, Cucumber, Carrot, Eggplant, Cauliflower, Horseradish, Morning Glory, Poplar, Walnut, Apple, Grape, Asparagus, Rice, Maize, Millet, Onion, Barley, Orchard grass, Oat, Rye, and Wheat. In addition, as is discussed below, expression constructs may be expressed in a transgenic plant to turn on the pathogen defense MAPK signal transduction pathway to enhance plant tolerance to its pathogen(s).

Materials for expressing these genes are available from a wide range of sources including the American Type Culture Collection (Rockland, Md.); or from any of a number seed companies, for example, W. Atlee Burpee Seed Co. (Warminster, Pa.), Park Seed Co. (Greenwood, S.C.), Johnny Seed Co. (Albion, Me.), or Northrup King Seeds (Harstville, S.C.). Descriptions and sources of useful host cells are also found in Vasil I. K., Cell Culture and Somatic Cell Genetics of Plants, Vol I, II, III Laboratory Procedures and Their Applications, Academic Press, New York, 1984; Dixon, R. A., Plant Cell Culture-A Practical Approach, IRL Press, Oxford University, 1985; Green et al., Plant Tissue and Cell Culture, Academic Press, New York, 1987; and Gasser and Fraley, Science 244: 1293, 1989.

The method of transformation or transfection and the choice of vehicle for expression of the MAPKKK polypeptide or its kinase domain (or a MAPKK or WRKY polypeptide) will depend on the host system selected. Transformation and transfection methods are described, e.g., in Ausubel et al. (supra); Weissbach and Weissbach, Methods for Plant Molecular Biology, Academic Press, 1989; Gelvin et al., Plant Molecular Biology Manual, Kluwer Academic Publishers, 1990; Kindle, K., Proc. Natl. Acad. Sci., U.S.A. 87: 1228, 1990; Potrykus, I., Annu. Rev. Plant Physiol. Plant Mol. Biology 42: 205, 1991; and BioRad (Hercules, Calif.) Technical Bulletin #1687 (Biolistic Particle Delivery Systems). Expression vehicles may be chosen from those provided, e.g., in Cloning Vectors: A Laboratory Manual (P. H. Pouwels et al., 1985, Supp. 1987); Gasser and Fraley (supra); Clontech Molecular Biology Catalog (Catalog 1992/93 Tools for the Molecular Biologist, Palo Alto, Calif.); and the references cited above. Other expression constructs are described by Fraley et al. (U.S. Pat. No. 5,352,605).

Most preferably, a MAPKKK polypeptide or its kinase domain (or a MAPKK or WRKY polypeptide) is produced by a stably-transfected plant cell line, a transiently-transfected plant cell line, or by a transgenic plant. A number of vectors suitable for stable transfection of plant cells or for the establishment of transgenic plants are available to the public; such vectors are described in Pouwels et al. (supra), Weissbach and Weissbach (supra), and Gelvin et al. (supra). Methods for constructing such cell lines are described in, e.g., Weissbach and Weissbach (supra), and Gelvin et al. (supra). Typically, plant expression vectors include (1) a cloned plant gene under the transcriptional control of 5′ and 3′ regulatory sequences and (2) a dominant selectable marker. Such plant expression vectors may also contain, if desired, a promoter regulatory region (for example, one conferring inducible or constitutive, pathogen- or wound-induced, environmentally- or developmentally-regulated, or cell- or tissue-specific expression), a transcription initiation start site, a ribosome binding site, an RNA processing signal, a transcription termination site, and/or a polyadenylation signal.

Once the desired nucleic acid sequence encoding a MAPKKK polypeptide or its kinase domain (or a MAPKK or WRKY polypeptide) is obtained as described above, it may be manipulated in a variety of ways known in the art. For example, where the sequence involves non-coding flanking regions, the flanking regions may be subjected to mutagenesis.

For example, the kinase domain sequence (or a MAPKKK polypeptide or kinase domain-containing fragment thereof), if desired, may be combined with other DNA sequences in a variety of ways. Such a sequence may be employed with all or part of the gene sequences normally associated with itself. In its component parts, a DNA sequence encoding a MAPKKK polypeptide or its kinase domain is combined in a DNA construct having a transcription initiation control region capable of promoting transcription and translation in a host cell.

In general, the constructs will involve regulatory regions functional in plants which provide for modified production of the regulator protein as discussed herein. The open reading frame coding for the regulator protein or functional fragment thereof will be joined at its 5′ end to a transcription initiation regulatory region such as the sequence naturally found in the 5′ upstream region of the MAPKKK polypeptide or its kinase domain; a MAPKK; or a WRKY polypeptide. Numerous other transcription initiation regions are available which provide for constitutive or inducible regulation.

For applications where developmental, cell, tissue, hormonal, or environmental expression is desired, appropriate 5′ upstream non-coding regions are obtained from other genes, for example, from genes regulated during meristem development, seed development, embryo development, or leaf development.

Regulatory transcript termination regions may also be provided in DNA constructs of this invention as well. Transcript termination regions may be provided by the DNA sequence encoding the MAPKKK polypeptide (or a MAPKK or WRKY polypeptide) or any convenient transcription termination region derived from a different gene source. The transcript termination region will contain preferably at least 1-3 kb of sequence 3′ to the structural gene from which the termination region is derived. Plant expression constructs having, for example, a MAPKKK protein kinase domain (e.g., the NPK1 kinase domain); a MAPKK polypeptide; or a WRKY polypeptide as the DNA sequence of interest for expression may be employed with a wide variety of plant life. Such genetically-engineered plants are useful for a variety of industrial and agricultural applications as discussed herein. Importantly, this invention is applicable to dicotyledons and monocotyledons, and will be readily applicable to any new or improved transformation or regeneration method.

An example of a useful plant promoter according to the invention is a caulimovirus promoter, for example, a cauliflower mosaic virus (CaMV) promoter. These promoters confer high levels of expression in most plant tissues, and the activity of these promoters is not dependent on virally encoded proteins. CaMV is a source for both the 35S and 19S promoters. In most tissues of transgenic plants, the CaMV 35S promoter is a strong promoter (see, e.g., Odell et al., Nature 313: 810, 1985). The CaMV promoter is also highly active in monocots (see, e.g., Dekeyser et al., Plant Cell 2: 591, 1990; Terada and Shimamoto, Mol. Gen. Genet. 220: 389, 1990). Moreover, activity of this promoter can be further increased (i.e., between 2-10 fold) by duplication of the CaMV 35S promoter (see e.g., Kay et al., Science 236: 1299, 1987; Ow et al., Proc. Nat. Acad. Sci., U.S.A. 84: 4870, 1987; and Fang et al., Plant Cell 1: 141, 1989). In addition, the a minimal 35S promoter may also be used as is described herein.

Other useful plant promoters include, without limitation, the nopaline synthase promoter (An et al., Plant Physiol. 88: 547, 1988) and the octopine synthase promoter (Fromm et al., Plant Cell 1: 977, 1989).

For certain applications, it may be desirable to produce the MAPKKK polypeptide or its kinase domain (or a MAPKK or WRKY polypeptide) in an appropriate tissue, at an appropriate level, or at an appropriate developmental time. For this purpose, there are an assortment of gene promoters, each with its own distinct characteristics embodied in its regulatory sequences, shown to be regulated in response to the environment, hormones, and/or developmental cues. These include gene promoters that are responsible for heat-regulated gene expression (see, e.g., Callis et al., Plant Physiol. 88: 965, 1988; Takahashi and Komeda, Mol. Gen. Genet. 219: 365, 1989; and Takahashi et al., Plant J. 2: 751, 1992), light-regulated gene expression (e.g., the Arabidopisis Cab2 photosynthetic, leaf specific promoter described by Mitra at el., Plant Mol. Biol. 12: 169-179, 1989; the pea rbcS-3A described by Kuhlemeier et al., Plant Cell 1: 471, 1989; the maize rbcS promoter described by Schäffner and Sheen, Plant Cell 3: 997, 1991; or the cholorphyll a/b-binding protein gene found in pea described by Simpson et al., EMBO J. 4: 2723, 1985), hormone-regulated gene expression (for example, the abscisic acid (ABA) responsive sequences from the Em gene of wheat described by Marcotte et al., Plant Cell 1: 969, 1989; the ABA-inducible HVA1 and HVA22, and rd29A promoters described for barley and Arabidopsis by Straub et al., Plant Cell 6: 617, 1994, Shen et al., Plant Cell 7: 295, 1995; and wound-induced gene expression (for example, of wunI described by Siebertz et al., Plant Cell 1: 961, 1989), organ-specific gene expression (for example, of the tuber-specific storage protein gene described by Roshal et al., EMBO J. 6: 1155, 1987; the 23-kDa zein gene from maize described by Schernthaner et al., EMBO J. 7: 1249, 1988; or the French bean β-phaseolin gene described by Bustos et al., Plant Cell 1: 839, 1989; the vegetative storage protein promoter (soybean vspB) described by Sadka et al (Plant Cell 6: 737-749, 1994)), cycling promoters (e.g., the Arabidopsis cdc2a promoter described by Hemerly et al., Proc. Natl. Acad. Sci., USA 89: 3295-3299, 1992), senescence-specific promoters (e.g., the Arabidopsis SAG12 promoter described by Gan et al, Science: 270, 1986-1988, 1995), seed-specific promoters (for example, endosperm-specific or embryo-specific promoters), or pathogen-inducible promoters (for example, PR-1 or β-1,3 glucanase promoters).

Plant expression vectors may also optionally include RNA processing signals, e.g, introns, which have been shown to be important for efficient RNA synthesis and accumulation (Callis et al., Genes and Dev. 1: 1183, 1987). The location of the RNA splice sequences can dramatically influence the level of transgene expression in plants. In view of this fact, an intron may be positioned upstream or downstream of a MAPKKK polypeptide or its kinase-domain encoding sequence (or a MAPKK polypeptide encoding sequence or a WRKY polypeptide encoding sequence) in the transgene to modulate levels of gene expression.

In addition to the aforementioned 5′ regulatory control sequences, the expression vectors may also include regulatory control regions which are generally present in the 3′ regions of plant genes (Thornburg et al., Proc. Natl. Acad. Sci., U.S.A. 84: 744, 1987; An et al., Plant Cell 1: 115, 1989). For example, the 3′ terminator region may be included in the expression vector to increase stability of the mRNA. One such terminator region may be derived from the PI-II terminator region of potato. In addition, other commonly used terminators are derived from the octopine or nopaline synthase signals.

The plant expression vector also typically contains a dominant selectable marker gene used to identify those cells that have become transformed. Useful selectable genes for plant systems include genes encoding antibiotic resistance genes, for example, those encoding resistance to hygromycin, kanamycin, bleomycin, G418, streptomycin, or spectinomycin. Genes required for photosynthesis may also be used as selectable markers in photosynthetic-deficient strains. Finally, genes encoding herbicide resistance may be used as selectable markers; useful herbicide resistance genes include the bar gene encoding the enzyme phosphinothricin acetyltransferase and conferring resistance to the broad spectrum herbicide Basta® (Hoechst A G, Frankfurt, Germany).

Efficient use of selectable markers is facilitated by a determination of the susceptibility of a plant cell to a particular selectable agent and a determination of the concentration of this agent which effectively kills most, if not all, of the transformed cells. Some useful concentrations of antibiotics for tobacco transformation include, e.g., 75-100 μg/mL (kanamycin), 20-50 μg/mL (hygromycin), or 5-10 μg/mL (bleomycin). A useful strategy for selection of transformants for herbicide resistance is described, e.g., by Vasil et al., supra.

It should be readily apparent to one skilled in the art of molecular biology, especially in the field of plant molecular biology, that the level of gene expression is dependent, not only on the combination of promoters, RNA processing signals, and terminator elements, but also on how these elements are used to increase the levels of selectable marker gene expression.

Plant Transformation

Upon construction of the plant expression vector, several standard methods are available for introduction of the vector into a plant host, thereby generating a transgenic plant. These methods include (1) Agrobacterium-mediated transformation (A. tumefaciens or A. rhizogenes) (see, e.g., Lichtenstein and Fuller, In: Genetic Engineering, vol 6, P W J Rigby, ed, London, Academic Press, 1987; and Lichtenstein, C. P., and Draper, J,. In: DNA Cloning, Vol II, D. M. Glover, ed, Oxford, IRI Press, 1985)), (2) the particle delivery system (see, e.g., Gordon-Kamm et al., Plant Cell 2: 603,1990); or BioRad Technical Bulletin 1687, supra), (3) microinjection protocols (see, e.g., Green et al., supra), (4) polyethylene glycol (PEG) procedures (see, e.g., Draper et al., Plant Cell Physiol. 23: 451, 1982; or e.g., Zhang and Wu, Theor. Appl. Genet. 76: 835, 1988), (5) liposome-mediated DNA uptake (see, e.g., Freeman et al., Plant Cell Physiol. 25: 1353, 1984), (6) electroporation protocols (see, e.g., Gelvin et al., supra; Dekeyser et al., supra; Fromm et al., Nature 319: 791, 1986; Sheen, Plant Cell 2: 1027, 1990; or Jang and Sheen, Plant Cell 6: 1665, 1994), and (7) the vortexing method (see, e.g., Kindle supra). The method of transformation is not critical to the invention. Any method which provides for efficient transformation may be employed. As newer methods are available to transform crops or other host cells, they may be directly applied.

The following is an example outlining one particular technique, an Agrobacterium-mediated plant transformation. By this technique, the general process for manipulating genes to be transferred into the genome of plant cells is carried out in two phases. First, cloning and DNA modification steps are carried out in E. coli, and the plasmid containing the gene construct of interest is transferred by conjugation or electroporation into Agrobacterium. Second, the resulting Agrobacterium strain is used to transform plant cells. Thus, for the generalized plant expression vector, the plasmid contains an origin of replication that allows it to replicate in Agrobacterium and a high copy number origin of replication functional in E. coli. This permits facile production and testing of transgenes in E. coli prior to transfer to Agrobacterium for subsequent introduction into plants. Resistance genes can be carried on the vector, one for selection in bacteria, for example, streptomycin, and another that will function in plants, for example, a gene encoding kanamycin resistance or herbicide resistance. Also present on the vector are restriction endonuclease sites for the addition of one or more transgenes and directional T-DNA border sequences which, when recognized by the transfer functions of Agrobacterium, delimit the DNA region that will be transferred to the plant.

In another example, plant cells may be transformed by shooting into the cell tungsten microprojectiles on which cloned DNA is precipitated. In the Biolistic Apparatus (Bio-Rad) used for the shooting, a gunpowder charge (22 caliber Power Piston Tool Charge) or an air-driven blast drives a plastic macroprojectile through a gun barrel. An aliquot of a suspension of tungsten particles on which DNA has been precipitated is placed on the front of the plastic macroprojectile. The latter is fired at an acrylic stopping plate that has a hole through it that is too small for the macroprojectile to pass through. As a result, the plastic macroprojectile smashes against the stopping plate, and the tungsten microprojectiles continue toward their target through the hole in the plate. For the instant invention the target can be any plant cell, tissue, seed, or embryo. The DNA introduced into the cell on the microprojectiles becomes integrated into either the nucleus or the chloroplast.

In general, transfer and expression of transgenes in plant cells are now routine practices to those skilled in the art, and have become major tools to carry out gene expression studies in plants and to produce improved plant varieties of agricultural or commercial interest.

Transgenic Plant Regeneration

Plant cells transformed with a plant expression vector can be regenerated, for example, from single cells, callus tissue, or leaf discs according to standard plant tissue culture techniques. It is well known in the art that various cells, tissues, and organs from almost any plant can be successfully cultured to regenerate an entire plant; such techniques are described, e.g., in Vasil supra; Green et al., supra; Weissbach and Weissbach, supra; and Gelvin et al., supra.

In one particular example, a cloned constitutively active kinase domain of a MAPKKK (or a MAPKKK polypeptide or a kinase-containing fragment thereof); a constitutively active MAPKK polypeptide; or a WRKY polypeptide; or any combination thereof, is placed under the control of the NOS promoter and the nopaline synthase terminator and carrying a selectable marker (for example, kanamycin resistance) is transformed into Agrobacterium. Transformation of leaf discs (for example, of tobacco or potato leaf discs), with vector-containing Agrobacterium is carried out as described by Horsch et al. (Science 227: 1229, 1985). Putative transformants are selected after a few weeks (for example, 3 to 5 weeks) on plant tissue culture media containing kanamycin (e.g. 100 μg/mL). Kanamycin-resistant shoots are then placed on plant tissue culture media without hormones for root initiation. Kanamycin-resistant plants are then selected for greenhouse growth. If desired, seeds from self-fertilized transgenic plants can then be sowed in a soil-less medium and grown in a greenhouse. Kanamycin-resistant progeny are selected by sowing surfaced sterilized seeds on hormone-free kanamycin-containing media. Analysis for the integration of the transgene is accomplished by standard techniques (see, for example, Ausubel et al. supra; Gelvin et al. supra).

Transgenic plants expressing the selectable marker are then screened for transmission of the transgene DNA by standard immunoblot and DNA detection techniques. Each positive transgenic plant and its transgenic progeny are unique in comparison to other transgenic plants established with the same transgene. Integration of the transgene DNA into the plant genomic DNA is in most cases random, and the site of integration can profoundly affect the levels and the tissue and developmental patterns of transgene expression. Consequently, a number of transgenic lines are usually screened for each transgene to identify and select plants with the most appropriate expression profiles.

Transgenic lines are evaluated for levels of transgene expression. Expression at the RNA level is determined initially to identify and quantitate expression-positive plants. Standard techniques for RNA analysis are employed and include PCR amplification assays using oligonucleotide primers designed to amplify only transgene RNA templates and solution hybridization assays using transgene-specific probes (see, e.g., Ausubel et al., supra). The RNA-positive plants are then analyzed for protein expression by Western immunoblot analysis using specific antibodies (see, e.g., Ausubel et al., supra). In addition, in situ hybridization and immunocytochemistry according to standard protocols can be done using transgene-specific nucleotide probes and antibodies, respectively, to localize sites of expression within transgenic tissue.

In addition, if desired, once the recombinant MAPKKK polypeptide or its kinase domain, a MAPKK polypeptide, or a WRKY polypeptide, or any combination thereof is expressed in any cell or in a transgenic plant (for example, as described above), it may be isolated, e.g., using affinity chromatography. In one example, an anti-MAPKKK polypeptide antibody (e.g., produced as described in Ausubel et al., supra, or by any standard technique) may be attached to a column and used to isolate the polypeptide. Lysis and fractionation of MAPKKK-producing cells prior to affinity chromatography may be performed by standard methods (see, e.g., Ausubel et al., supra). Once isolated, the recombinant protein can, if desired, be further purified, for example, by high performance liquid chromatography (see, e.g., Fisher, Laboratory Techniques In Biochemistry And Molecular Biology, eds., Work and Burdon, Elsevier, 1980).

Engineering Disease Resistance

As discussed above, plasmid constructs designed for the expression of constitutively active MAPKKKs or MAPKKs or expression of a WRKY gene product are useful, for example, for activating plant defense pathways that confer anti-pathogenic properties to a transgenic plant. Such genes are isolated from a host plant and may be engineered for expression in the same plant, a closely related species, or a distantly related plant species. For example, cruciferous genes may be engineered for constitutive low level expression and then transformed into an Arabidopsis host plant. Alternatively, the isolated cruciferous gene may be engineered for expression in other cruciferous plants, such as the Brassicas (for example, broccoli, cabbage, and cauliflower). Similarly, the MAPKKK, MAPKK, or WRKY is useful for expression in related solanaceous plants, such as tomato, potato, and pepper. To achieve pathogen resistance, it is important to express a constitutively active MAPKKK, MAPKK or a WRKY protein at an effective level. Evaluation of the level of pathogen protection conferred to a plant by ectopic expression of such polypeptides is determined according to conventional methods and assays.

In one working example, constitutive ectopic expression of the ANP1 or MEKKK gene in Russet Burbank potato is used to control Phytophthora infestans infection. In one particular example, a plant expression vector is constructed that contains the appropriate cDNA sequence expressed under the control of the enhanced CaMV 35S promoter as described by McPherson and Kay (U.S. Pat. No. 5,359,142). This expression vector is then used to transform Russet Burbank according to the methods described in Fischhoff et al. (U.S. Pat. No. 5,500,365). To assess resistance to fungal infection, transformed Russet Burbank and appropriate controls are grown to approximately eight-weeks-old, and leaves (for example, the second or third from the top of the plant) are inoculated with a mycelial suspension of P. infestans. Plugs of P. infestans mycelia are inoculated on each side of the leaf midvein. Plants are subsequently incubated in a growth chamber at 27° C. with constant fluorescent light.

Leaves of transformed Russet Burbank and control plants are then evaluated for resistance to P. infestans infection according to conventional experimental methods. For this evaluation, the number of lesions per leaf and percentage of leaf area infected are recorded every twenty-four hours for seven days after inoculation. From these data, levels of resistance to P. infestans are determined. Transformed potato plants that express an constitutively active ANP1 or MEKK1 or both having an increased level of resistance to P. infestans relative to control plants are taken as being useful in the invention.

Alternatively, to assess resistance at the whole plant level, transformed and control plants are transplanted to potting soil containing an inoculum of P. infestans. Plants are then evaluated for symptoms of fungal infection (for example, wilting or decayed leaves) over a period of time lasting from several days to weeks. Again, transformed potato plants expressing the ANP1 or MEKK1 or both having an increased level of resistance to the fungal pathogen, P. infestans, relative to control plants are taken as being useful in the invention.

In another working example, expression of an MAPKKK, MAPKK, or WRKY in tomato is used to control bacterial infection, for example, to Pseudomonas syringae. Specifically, a plant expression vector is constructed that contains the cDNA sequence of one or more of the genes encoding these polypeptides is expressed under the control of the enhanced CaMV 35S promoter as described by McPherson and Kay, supra. This expression vector is then used to transform tomato plants according to the methods described in Fischhoff et al., supra. To assess resistance to bacterial infection, transformed tomato plants and appropriate controls are grown, and their leaves are inoculated with a suspension of P. syringae according to standard methods, for example, those described herein. Plants are subsequently incubated in a growth chamber, and the inoculated leaves are subsequently analyzed for signs of disease resistance according to standard methods. For example, the number of chlorotic lesions per leaf and percentage of leaf area infected are recorded and evaluated after inoculation. From a statistical analysis of these data, levels of resistance to P. syringae are determined. Transformed tomato plants that express an MAPKKK, MAPKK, or WRKY gene having an increased level of resistance to P. syringae relative to control plants are taken as being useful in the invention.

In still another working example, expression of an MAPKKK, MAPKK, or WRKY gene of rice is used to control fungal diseases, for example, the infection of tissue by Magnaporthe grisea, the cause of rice blast. In one particular approach, a plant expression vector is constructed that contains the cDNA sequence of the rice MAPKKK, MAPKK, or WRKY gene that is constitutively expressed under the control of the rice actin promoter described by Wu et al. (WO 91/09948). This expression vector is then used to transform rice plants according to conventional methods, for example, using the methods described in Hiei et al. (Plant Journal 6:271-282, 1994). To assess resistance to fungal infection, transformed rice plants and appropriate controls are grown, and their leaves are inoculated with a mycelial suspension of M. grisea according to standard methods. Plants are subsequently incubated in a growth chamber and the inoculated leaves are subsequently analyzed for disease resistance according to standard methods. For example, the number of lesions per leaf and percentage of leaf area infected are recorded and evaluated after inoculation. From a statistical analysis of these data, levels of resistance to M grisea are determined. Transformed rice plants that express a gene having an increased level of resistance to M. grisea relative to control plants are taken as being useful in the invention.

Use

The invention described herein is useful for a variety of agricultural and commercial purposes including, but not limited to, improving pathogen resistance, increasing crop yields, improving crop and ornamental quality, and reducing agricultural production costs. In particular, ectopic expression of a kinase domain of a MAPKKK polypeptide (or a MAPKKK polypeptide or a kinase domain-containing fragment thereof); a MAPKK polypeptide, or a WRKY polypeptide in a plant cell provides resistance to a plant pathogen and can be used to protect plants from pathogens that reduce plant productivity and viability. The invention therefore provides pathogen and disease resistance to plants, especially crop plants, most especially crop plants such as tomato, potato, cotton, pepper, maize, wheat, rice, and legumes such as soybean, or any crop plant that is susceptible to pathogens. For example, transgenic maize and soybean may be genetically engineered to express a kinase domain of a MAPKKK (e.g., NPK1 or an ANP such as ANP1, ANP2, or ANP3), a MAPKK polypeptide, or a WRKY polypeptide, or any combination thereof according to standard methods, such as those described in Adams et al. (U.S. Pat. No. 5,550,318) and Collins et al. (U.S. Pat. No. 5,024,944). Methods for transforming wheat with such genes are described in Fry et al. (U.S. Pat. No. 5,631,152).

### Other Embodiments

The invention further includes the use of analogs of any naturally-occurring MAPKKK polypeptide, a MAPKK polypeptide, or a WRKY polypeptide. Analogs can differ from the naturally-occurring kinase domain by amino acid sequence differences, by post-translational modifications, or by both. Analogs of the invention will generally exhibit at least 40%, more preferably 50%, and most preferably 60% or even having 70%, 80%, or 90% identity with all or part of a naturally-occurring kinase domain amino acid sequence. The length of sequence comparison is at least 15 amino acid residues, preferably at least 25 amino acid residues, and more preferably more than 35 amino acid residues. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring kinase domain polypeptide by alterations in primary sequence. These include genetic variants, both natural and induced (for example, resulting from random mutagenesis by irradiation or exposure to ethyl methylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., or amino acids.

In addition, the invention also includes kinase domain fragments. As used herein, the term “fragment,” means at least 50 contiguous amino acids, preferably at least 130 contiguous amino acids, more preferably at least 160 contiguous amino acids, and most preferably at least 190 to 230 or more contiguous amino acids. Fragments of kinase domain polypeptides can be generated by methods known to those skilled in the art or may result from normal protein processing (e.g., removal of amino acids from the nascent polypeptide that are not required for biological activity or removal of amino acids by alternative mRNA splicing or alternative protein processing events). In preferred embodiments, a kinase domain fragment (e.g., a fragment of NPK1, ANP1, ANP2, or ANP3) is capable of activating the transcription of a plant defense gene. Methods for evaluating such activity are described herein.

All publications and patent applications mentioned in this specification are herein incorporated by reference.

Other embodiments are with the claims.

1. A method of enhancing resistance to a plant pathogen in a plant, said method comprising:
(a) providing a transgenic plant cell that expresses an isolated DNA molecule encoding a kinase domain of a MAPKK polypeptide; and
(b) regenerating a plant from said plant cell wherein said isolated DNA molecule is expressed in said plant, and wherein said plant has enhanced resistance to a plant pathogen compared to a corresponding untransformed plant.

2. The method of claim 1, wherein said plant is a dicot.

3. The method of claim 2, wherein said dicot is a crucifer.

4. The method of claim 3, wherein said crucifer is Arabidopsis.

5. The method of claim 1, wherein said plant is a monocot.

6. The method of claim 1, wherein said kinase domain is constitutively active.

7. The method of claim 1, wherein said MAPKK polypeptide is MKK4.

8. The method of claim 1, wherein said MAPKK polypeptide is MKK5.

9. The method of claim 1, wherein said MAPKK polypeptide activates a gene involved in pathogen defense.

10. The method of claim 1, wherein said MAPKK polypeptide activates the PAL1, GST1, WRKY29, or PR1 gene promoters.

11. A method of enhancing resistance to a plant pathogen in a plant, said method comprising:
(a) providing a plant cell that expresses an isolated DNA molecule encoding a kinase domain of a MAPKKK polypeptide; and
(b) regenerating a plant from said plant cell wherein said isolated DNA molecule is expressed in said plant, and wherein said plant has enhanced resistance to a plant pathogen compared to a corresponding untransformed plant.

12. The method of claim 11, wherein said plant is a dicot.

13. The method of claim 12, wherein said dicot is a crucifer.

14. The method of claim 13, wherein said crucifer is Arabidopsis.

15. The method of claim 11, wherein said plant is a monocot.

16. The method of claim 11, wherein said kinase domain is constitutively active.

17. The method of claim 11, wherein said MAPKKK polypeptide is MEKK1.

18. The method of claim 11, wherein said MAPKKK polypeptide is ANP1.

19. The method of claim 11, wherein said MAPKKK polypeptide activates a gene involved in pathogen defense.

20. The method of claim 11, wherein said MAPKKK polypeptide activates the PAL1, GST1, WRKY29, or PR1 gene promoters.

21. A method of enhancing resistance to a plant pathogen in a plant, said method comprising the steps of:
(a) providing a plant cell that expresses an isolated DNA molecule encoding a polypeptide comprising a polypeptide having substantial identity to a WRKY polypeptide; and
(b) regenerating a plant from said plant cell wherein said isolated DNA molecule is expressed in said plant, and wherein said plant has enhanced resistance to a plant pathogen compared to a corresponding untransformed plant.

22. The method of claim 21, wherein said plant is a dicot.

23. The method of claim 22, wherein said dicot is a crucifer.

24. The method of claim 23, wherein said crucifer is Arabidopsis.

25. The method of claim 21, wherein said plant is a monocot.

26. The method of claim 21, wherein said WRKY polypeptide induces its own expression.

27. An isolated nucleic acid molecule having a nucleotide sequence for a promoter that is capable of initiating pathogen-inducible transcription in a plant cell, wherein said nucleotide sequence is selected from the group consisting of:
a) a nucleotide sequence comprising the sequence set forth in FIGS. 15 or 16;
b) a nucleotide sequence comprising at least 40 contiguous nucleotides of the sequence set forth in FIGS. 15 or 16; and
c) a nucleotide sequence that has at least about 70% sequence identity to a sequence set forth in a) or b).

28. A DNA construct comprising a nucleotide sequence of claim 27 operably linked to a heterologous nucleotide sequence of interest.

29. A vector comprising the DNA construct of claim 27.

30. A host cell having stably incorporated in its genome the DNA construct of claim 27.

31. A method for expressing a heterologous nucleotide sequence in a plant, said method comprising transforming a plant cell with a DNA construct comprising said heterologous nucleotide sequence operably linked to a promoter that is capable of initiating transcription in a plant cell and regenerating a stably transformed plant from said plant cell, wherein said promoter comprises a nucleotide sequence selected from the group consisting of:
a) a nucleotide sequence comprising the sequence set forth in FIGS. 15 or 16;
b) a nucleotide sequence comprising at least 40 contiguous nucleotides of the sequence set forth in FIGS. 15 or 16; and
c) a nucleotide sequence that has at least about 70% sequence identity to a sequence set forth in a) or b).

32. The method of claim 31, wherein said plant is a dicot.

33. The method of claim 32, wherein said dicot is a crucifer.

34. The method of claim 33, wherein said crucifer is Arabidopsis.

35. The method of claim 31, wherein said plant is a monocot.

36. A plant cell stably transformed with a DNA construct comprising a heterologous nucleotide sequence operably linked to a promoter that is capable of initiating transcription in said plant cell, wherein said promoter comprises a nucleotide sequence selected from the group consisting of:
a) a nucleotide sequence comprising the sequence set forth in FIGS. 15 or 16;
b) a nucleotide sequence comprising at least 40 contiguous nucleotides of the sequence set forth in FIGS. 15 or 16; and
c) a nucleotide sequence that has at least about 70% sequence identity to a sequence set forth in a) or b).

37. The plant of claim 36, wherein said plant is a dicot.

38. The plant of claim 37, wherein said dicot is a crucifer.

39. The plant of claim 28, wherein said crucifer is Arabidopsis.

40. The plant of claim 36, wherein said plant is a monocot.
